(19) United States
(12) Patent Application Publication
(10) Pub. No.: US 2010/0061994 A1
(43) Pub. Date: Mar. 11, 2010

(54) MEDICAL USES OF 39-DESMETHOXYRAPAMYCIN AND ANALOGUES THEREOF

(76) Inventors: Rose Mary Sheridan, Essex (GB); Mingqiang Zhang, Essex (GB); Matthew Alan Gregory, Essex (GB)

Correspondence Address: WYETH LLC;PATENT LAW GROUP, 5 GIRALDA FARMS, MADISON, NJ, 07940, US

(21) Appl. No.: 11/908,367

(22) PCT Filed: Mar. 10, 2006
(86) PCT No.: PCT/GB2006/000834

§ 371 Date: Mar. 24, 2009

Related U.S. Application Data
(63) Continuation-in-part of application No. 11/097,605, filed on Apr. 1, 2005, now Pat. No. 7,183,289.

(30) Foreign Application Priority Data
Mar. 11, 2005 (GB) 0504995.2
Nov. 9, 2005 (GB) 0522829.1

(51) Int. Cl. A61K 39/395; A61K 31/4353; A61K 31/4985; A61K 31/704; A61K 38/14; A61K 31/66; A61K 38/08; A61P 25/00; A61P 35/00

(52) U.S. Cl. 424/143.1; 514/291; 514/249; 514/34; 514/8; 514/110; 514/15

(57) ABSTRACT

The present invention relates to medical uses of 39-desmethoxyrapamycin analogues.

### BACKGROUND OF THE INVENTION

Rapamycin (sirolimus) (FIG. 1) is a lipophilic macrolide produced by Streptomyces hygroscopicus NRRL 5491 (Sehgal et al., 1975; Vézina et al., 1975; U.S. Pat. No. 3,929,992; U.S. Pat. No. 3,993,749) with a 1,2,3-tricarbonyl moiety linked to a pipecolic acid lactone (Paiva et al., 1991). For the purpose of this invention rapamycin is described by the numbering convention of McAlpine et al. (1991) in preference to the numbering conventions of Findlay et al. (1980) or Chemical Abstracts (11^{th} Cumulative Index, 1982-1986 p60719CS).

Rapamycin has significant therapeutic value due to its wide spectrum of biological activities (Huang et al., 2003). The compound is a potent inhibitor of the mammalian target of rapamycin (mTOR), a serine-threonine kinase downstream of the phosphatidylinositol 3-kinase (PI3K)/Akt (protein kinase B) signalling pathway that mediates cell survival and proliferation. This inhibitory activity is gained after rapamycin binds to the immunophilin FK506 binding protein 12 (FKBP12) (Dumont, F. J. and Q. X. Su, 1995). In T cells rapamycin inhibits signalling from the IL-2 receptor and subsequent autoproliferation of the T cells resulting in immunosuppression. Rapamycin is marketed as an immunosuppressant for the treatment of organ transplant patients to prevent graft rejection (Huang et al, 2003). In addition to immunosuppression, rapamycin has potential therapeutic use in the treatment of a number of diseases, for example, cancer, cardiovascular diseases such as restenosis, autoimmune diseases such as multiple sclerosis, rheumatoid arthritis, fungal infection and neurodegenerative diseases such as Alzheimer's disease, Parkinson's disease and Huntington's disease.

Despite its utility in a variety of disease states rapamycin has a number of major drawbacks. Firstly it is a substrate of cell membrane efflux pump P-glycoprotein (P-gp, LaPlante et al, 2002, Crowe et al, 1999) which pumps the compound out of the cell making the penetration of cell membranes by rapamycin poor. This causes poor absorption of the compound after dosing. In addition, since a major mechanism of multi-drug resistance of cancer cells is via cell membrane efflux pump, rapamycin is less effective against multi-drug resistance (MDR) cancer cells. Secondly rapamycin is extensively metabolised by cytochrome P450 enzymes (Lampen et al, 1998). Its loss at hepatic first pass is high, which contributes further to its low oral bioavailability. The role of CYP3A4 and P-gp in the low bioavailability of rapamycin has been confirmed in studies demonstrating that administration of CYP3A4 and/or P-gp inhibitors decreased the efflux of rapamycin from CYP3A4-transfected Caco-2 cells (Cummins et al, 2004) and that administration of CYP3A4 inhibitors decreased the small intestinal metabolism of rapamycin (Lampen et al, 1998). The low oral bioavailability of rapamycin causes significant inter-individual variability resulting in inconsistent therapeutic outcome and difficulty in clinical management (Kuhn et al, 2001, Crowe et al, 1999).

Therefore, there is a need for the development of novel rapamycin-like compounds that are not substrates of P-gp, that may be metabolically more stable and therefore may have improved bioavailability. When used as anticancer agents, these compounds may have better efficacy against MDR cancer cells, in particular against P-gp-expressing cancer cells.

A range of synthesised rapamycin analogues using the chemically available sites of the molecule has been reported. The description of the following compounds was adapted to the numbering system of the rapamycin molecule described in FIG. 1. Chemically available sites on the molecule for derivatisation or replacement include C40 and C28 hydroxyl groups (e.g. U.S. Pat. No. 5,665,772; U.S. Pat. No. 5,362,718), C39 and C16 methoxy groups (e.g. WO 96/41807; U.S. Pat. No. 5,728,710), C32, C26 and C9 keto groups (e.g. U.S. Pat. No. 5,378,836; U.S. Pat. No. 5,138,051; U.S. Pat. No. 5,665,772). Hydrogenation at C17, C19 and/or C21, targeting the triene, resulted in retention of antifungal activity but relative loss of immunosuppression (e.g. U.S. Pat. No. 5,391,730; U.S. Pat. No. 5,023,262). Significant improvements in the stability of the molecule (e.g. formation of oximes at C32, C40 and/or C28, U.S. Pat. No. 5,563,145, U.S. Pat. No. 5,446,048), resistance to metabolic attack (e.g. U.S. Pat. No. 5,912,253), bioavailability (e.g. U.S. Pat. No. 5,221,670; U.S. Pat. No. 5,955,457; WO 98/04279) and the production of prodrugs (e.g. U.S. Pat. No. 6,015,815; U.S. Pat. No. 5,432,183) have been achieved through derivatisation.

Two of the most advanced rapamycin derivatives in clinical development are 40-O-(2-hydroxy)ethyl-rapamycin (RAD001, Certican, everolimus) a semi-synthetic analogue of rapamycin that shows immunosuppressive pharmacological effects (Sedrani, R. et al, 1998; Kirchner et al., 2000; U.S. Pat. No. 5,665,772) and 40-O-[2,2-bis(hydroxymethyl)propionyloxy]rapamycin, CCI-779 (Wyeth-Ayerst) an ester of rapamycin which inhibits cell growth in vitro and inhibits tumour growth in vivo (Yu et al., 2001). CCI-779 is currently in various clinical trials as a potential anticancer drug. A recent publication of CCI-779 phase II study in patients with recurrent glioblastoma multiforme (Chang, et al., 2005) suggests the low efficacy of this drug in these patients may be due to its poor penetration of blood-brain barrier. Studies investigating the pharmacokinetics of RAD001 have shown that, similarly to rapamycin, it is a substrate for P-gp (Crowe at al, 1999, LaPlante at al, 2002).

Despite their close structural similarity to rapamycin the compounds of the invention displays a surprisingly different pharmacological profile. In particular they show significantly increased cell membrane permeability and decreased efflux in comparison with rapamycin, and they are not a substrate for P-gp. Additionally, 39-desmethoxyrapamycin shows more potent activity against multi-drug resistant and P-gp-expressing cancer cell lines than rapamycin. When compared with rapamycin 39-desmethoxyrapamycin shows a significantly different inhibitory profile against the NCI 60 cell line panels.

Additionally, 39-desmethoxyrapamycin analogues show a significantly different pharmacokinetic profile compared to rapamycin and the leading derivatives in clinical trials. Unexpectedly, 39-desmethoxyrapamycin analogues show an increased ability to cross the blood brain barrier and therefore demonstrate improved availability in the brain.

Therefore, the present invention provides for the medical use of 39-desmethoxyrapamycin analogues, these rapamycin analogues have significantly altered pharmacokinetics, improved ability to cross the blood brain barrier, improved metabolic stability, improved cell membrane permeability, a decreased rate of efflux and a different tumour cell inhibitory profile to rapamycin. These compounds are useful in medicine, in particular for the treatment of cancer and/or B-cell malignancies, in the induction or maintenance of immunosuppression, the stimulation of neuronal regeneration or the treatment of fungal infections, transplantation rejection, graft vs. host disease, autoimmune disorders, diseases of inflammation vascular disease and fibrotic diseases. The present invention particularly provides for the use of 39-desmethoxyrapamycin in the treatment of cancer and/or B-cell malignancies.

Rapamycin has been demonstrated to stimulate autophagy (Raught et al., 2001). Impaired autophagy or the dysregulation of autophagy has been implicated in a number of disorders including Alzheimer's disease, Parkinson's disease, Huntington's disease and prion diseases (including Creutzfeldt-Jacob disease) suggesting that manipulation of this pathway may prove beneficial in these diseases. A recent in vitro study demonstrated that administration 2D of rapamycin was able to reduce the appearance of aggregates and cell death associated with poly(Q) and poly(A) expansions in transfected COS-7 cells. (Ravikumar et al, 2002). Therefore, if rapamycin was able to cross the blood brain barrier these results indicate that it would make a suitable candidate for the treatment of Huntington's disease and other related disorders. This suggests that there is a need for the development of rapamycin analogues which are able to cross the blood brain barrier.

Hyperphosphorylation of the microtubule-associated protein tau and its subsequent aggregation into insoluble paired helical filaments which form intracellular “tangles” is one of the characteristic hallmarks of Alzheimer's disease and the accumulation of this neurofibrillary pathology and the associated neuronal cell death is closely related to the cognitive decline. A recent study by An et al (2003), demonstrated that activated p70 S6 kinase is co-distributed with neurofibrillary pathology in Alzheimer's brains and in particular activated p70 S6 kinase was obviously increased in neurons before the development of tangles (An et al., 2003). In an in vitro assay where zinc sulphate administration results in the activation of p70 S6 kinase and increased levels of total, normal and hyperphosphorylated tau, pre-treatment of the cells with rapamycin was shown reduce p70 S6 kinase activation three-fold and significantly reduce the levels of total, normal and hyperphosphorylated tau. Therefore, these results indicate that administration of rapamycin or rapamycin analogues may be of benefit in reducing the neurofibrillary pathology of Alzheimer's disease, provided that the compounds are able to reach the site of action.

Additionally, it has been reported that rapamycin increases neuritic outgrowth and neuronal survival in several in vitro and in vivo models (Avramut and Achim, 2002) indicating that rapamycin and analogues thereof may be of use in treating disorders where neuronal regeneration may be of significant therapeutic benefit. However, this utility is dependent on it being able to reach the site of action and therefore rapamycin analogues with an improved ability to cross the blood brain barrier would be particularly preferred.

The present invention provides the novel and surprising use of 39-desmethoxyrapamycin analogues in medicine, in particular the use of 39-desmethoxyrapamycin, particularly in the treatment of cancer or B-cell malignancies, in the induction or maintenance of immunosuppression, the stimulation of neuronal regeneration or the treatment of fungal infections, transplantation rejection, graft vs. host disease, autoimmune disorders, neurodegenerative conditions, diseases of inflammation vascular disease and fibrotic diseases. In particular the present invention provides for the use of 39-desmethoxyrapamycin analogues in the treatment of cancer and B-cell malignancies. In a preferred embodiment, the present invention provides for the use of 39-desmethoxyrapamycin analogues in the treatment of neurological or neurodegenerative disorders. In a further preferred embodiment, the present invention provides for the use of 39-desmethoxyrapamycin analogues in the treatment of brain tumours, in particular glioblastoma multiforme. In a specific aspect of the present invention, the 39-desmethoxyrapamycin analogue is 39-desmethoxyrapamycin.

### SUMMARY OF THE INVENTION

The present invention relates to the medical use of 39-desmethoxyrapamycin analogues, in particular 39-desmethoxyrapamycin, particularly in the treatment of cancer and/or B-cell malignancies, the induction or maintenance of immunosuppression, the treatment of transplantation rejection, graft vs. host disease, autoimmune disorders, neurodegenerative conditions, diseases of inflammation, vascular disease and fibrotic diseases, the stimulation of neuronal regeneration or the treatment of fungal infections. In particular this invention relates to the use of 39-desmethoxyrapamycin analogues for the treatment of cancer and B-cell malignancies. In a specific embodiment the present invention relates to the use of 39-desmethoxyrapamycin in the treatment of cancer and B-cell malignancies. The present invention also specifically provides for the use of 39-desmethoxyrapamycin analogues in the treatment of brain tumour(s) or neurodegenerative conditions. In a specific embodiment, the present invention provides for the use of 39-desmethoxyrapamycin in the treatment of brain tumour(s) or neurodegenerative conditions. The present invention also specifically provides for the use of 39-desmethoxyrapamycin analogues in the treatment of neurodegenerative conditions. In particular the present invention provides for the use of 39-desmethoxyrapamycin in the treatment in neurodegenerative conditions.

### Definitions

The articles “a” and “an” are used herein to refer to one or to more than one (i.e. at least one) of the grammatical objects of the article. By way Of example “an analogue” means one analogue or more than one analogue.

As used herein, the term “autoimmune disorder(s)” includes, without limitation: systemic lupus erythrematosis (SLE), rheumatoid arthritis, myasthenia gravis and multiple sclerosis.

As used herein, the term “diseases of inflammation” includes, without limitation: psoriasis, dermatitis, eczema, seborrhoea, inflammatory bowel disease (including but not limited to ulcerative colitis and Crohn's disease), pulmonary inflammation (including asthma, chronic obstructive pulmonary disease, emphysema, acute respiratory distress syndrome and bronchitis), rheumatoid arthritis and eye uveitis.

As used herein, the term “cancer” refers to a malignant or benign growth of cells in skin or in body organs, for example but without limitation, breast, prostate, lung, kidney, pancreas, brain, stomach or bowel. A cancer tends to infiltrate into adjacent tissue and spread (metastasise) to distant organs, for example to bone, liver, lung or the brain. As used herein the term cancer includes both metastatic tumour cell types, such as but not limited to, melanoma, lymphoma, leukaemia, fibrosarcoma, rhabdomyosarcoma, and mastocytoma and types of tissue carcinoma, such as but not limited to, colorectal cancer, prostate cancer, small cell lung cancer and non-small cell lung cancer, breast cancer, pancreatic cancer, bladder cancer, renal cancer, gastric cancer, gliobastoma, primary liver cancer and ovarian cancer. The term also specifically encompasses brain tumour(s) as described more fully below.

As used herein the term “brain tumour(s)” refers to a malignant or benign growth of cells in the brain, it includes primary and secondary (metastatic) tumours. Primary brain tumours include, without limitation, gliomas (e.g. glioblastoma multiforme, astrocytoma, brain stem glioma, ependymoma and oligodendroglioma), medulloblastoma, meningioma, schwannoma (or acoustic neuroma), craniopharyngioma, germ cell tumor of the brain (e.g. germinoma), or pineal region tumor. The term “brain cancer” is also used to describe the above set of disorders and these terms are used interchangeably herein.

As used herein the term “B-cell malignancies” includes a group of disorders that include chronic lymphocytic leukaemia (CLL), multiple myeloma, and non-Hodgkin's lymphoma (NHL). They are neoplastic diseases of the blood and blood forming organs. They cause bone marrow and immune system dysfunction, which renders the host highly susceptible to infection and bleeding.

As used herein, the term “vascular disease” includes, without limitation: hyperproliferative vascular disorders (e.g. restenosis and vascular occlusion), graft vascular atherosclerosis, cardiovascular disease, cerebral vascular disease and peripheral vascular disease (e.g. coronary artery disease, arteriosclerosis, atherosclerosis, nonatheromatous arteriosclerosis or vascular wall damage). It is also used to refer to diseases involving the neogenesis or proliferation of blood vessels in the eye, in particular choroidal neovascularisation.

As used herein the terms “neuronal regeneration” refers to the stimulation of neuronal cell growth and includes neurite outgrowth and functional recovery of neuronal cells. Diseases and disorders where neuronal regeneration may be of significant therapeutic benefit include, but are not limited to, Alzheimer's disease, Parkinson's disease, Huntington's chorea (disease), amyotrophic lateral sclerosis, trigeminal neuralgia, glossopharyngeal neuralgia, Bell's palsy, muscular dystrophy, stroke, progressive muscular atrophy, progressive bulbar inherited muscular atrophy, cervical spondylosis, Gullain-Barre syndrome, dementia, peripheral neuropathies and peripheral nerve damage, whether caused by physical injury (e.g. spinal cord injury or trauma, sciatic or facial nerve lesion or injury) or a disease state (e.g. diabetes).

As used herein, the terms “medical condition resulting from neural injury or disease” includes without limitation, neurodegenerative condition(s), brain tumour(s), infection or inflammation of the brain and other conditions which may lead to death or dysfunction of nervous or glial cells or tissues.

As used herein the term “neurodegenerative condition(s)” includes, without limitation, Alzheimer's disease, Parkinson's disease, Huntington's disease, amyotrophic lateral sclerosis (ALS), (oculopharyngeal) muscular dystrophy, (including oculopharyngeal muscular dystrophy), multiple sclerosis, prion diseases (e.g. Creutzfeldt-Jacob disease (CJD)), Pick's disease, Lewy body dementia (or Lewy body disease) and/or motor neurone disease.

As used herein, the term “medical condition affecting the central nervous which requires the medicament to cross the blood-brain barrier” includes without limitation medical conditions resulting from neural injury or diseases, and any other condition for which the access of the medicament to the neuronal cells is required for effective therapy.

As used herein the term “fibrotic diseases” refers to diseases associated with the excess production of the extracellular matrix and includes (without limitation) sarcoidosis, keloids, glomerulonephritis, end stage renal disease, liver fibrosis (including but not limited to cirrhosis, alcohol liver disease and steato-heptatitis), chronic graft nephropathy, surgical adhesions, vasculopathy, cardiac fibrosis, pulmonary fibrosis (including but not limited to idiopathic pulmonary fibrosis and cryptogenic fibrosing alveolitis), macular degeneration, retinal and vitreal retinopathy and chemotherapy or radiation-induced fibrosis.

As used herein, the term “graft vs. host disease” refers to a complication that is observed after allogeneic stem cell/bone marrow transplant. It occurs when infection-fighting cells from the donor recognize the patient's body as being different or foreign. These infection-fighting cells then attack tissues in the patient's body just as if they were attacking an infection. GvHD is categorized as acute when it occurs within the first 100 days after transplantation and chronic if it occurs more than 100 days after transplantation. Tissues typically involved include the liver, gastrointestinal tract and skin. Chronic graft vs. host disease occurs approximately in 10-40 percent of patients after stem cell/bone marrow transplant.

As used herein, the term “bioavailability” refers to the degree to which or rate at which a drug or other substance is absorbed or becomes available at the site of biological activity after administration. This property is dependent upon a number of factors including the solubility of the compound, rate of absorption in the gut, the extent of protein binding and metabolism etc. Various tests for bioavailability that would be familiar to a person of skill in the art are described herein (see also Trepanier et al., 1998, Gallant-Haidner et al, 2000).

As used herein the term “cancer or B-cell malignancy resistant to one or more existing anticancer agent(s)” refers to cancers or B-cell malignancies for which at least one typically used therapy is ineffective. These cancers are characterised by being able to survive after the administration of at least one neoplastic agent where the normal cell counterpart (i.e., a growth regulated cell of the same origin) would either show signs of cell toxicity, cell death or cell quiescence (i.e., would not divide). In particular, this includes MDR cancers or B-cell malignancies, particular examples are cancers and B-cell malignancies which express high levels of P-gp. The identification of such resistant cancers or B-cell malignancies is within the ability and usual activities of a physician or other similarly skilled person.

As used herein the term “39-desmethoxyrapamycin analogues” refers to a compound according to formula (I) below, or a pharmaceutically acceptable salt thereof.

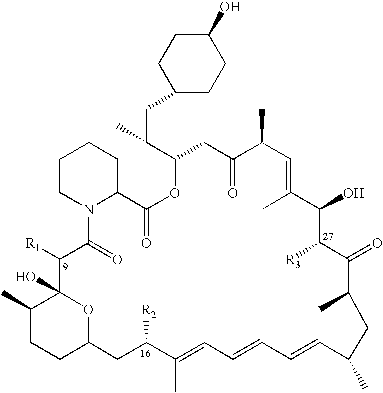

wherein, R₁ represents (H,H) or ═O, and R₂ and R₃ each independently represents H, OH or OCH₃. These compounds are also referred to as the “compounds of the invention” and these terms are used interchangeably in the present application.

In the present application the term “39-desmethoxyrapamycin analogue” includes 39-desmethoxyrapamycin itself.

As used herein, the term “39-desmethoxyrapamycin” refers to a compound according to formula (I) above, or a pharmaceutically acceptable salt thereof, wherein R₁ represents ═O, and R₂ and R₃ each represent OCH₃.

The pharmaceutically acceptable salts of 39-desmethoxyrapamycin analogues include conventional salts formed from pharmaceutically acceptable inorganic or organic acids or bases as well as quaternary ammonium acid addition salts. More specific examples of suitable acid salts include hydrochloric, hydrobromic, sulfuric, phosphoric, nitric, perchloric, fumaric, acetic, propionic, succinic, glycolic, formic, lactic, maleic, tartaric, citric, palmoic, malonic, hydroxymaleic, phenylacetic, glutamic, benzoic, salicylic, fumaric, toluenesulfonic, methanesulfonic, naphthalene-2-sulfonic, benzenesulfonic hydroxynaphthoic, hydroiodic, malic, steroic, tannic and the like. Other acids such as oxalic, while not in themselves pharmaceutically acceptable, may be useful in the preparation of salts useful as intermediates in obtaining the compounds of the invention and their pharmaceutically acceptable salts. More specific examples of suitable basic salts include sodium, lithium, potassium, magnesium, aluminum, calcium, zinc, N,N′-dibenzylethylenediamine, chloroprocaine, choline, diethanolamine, ethylenediamine, N-methylglucamine and procaine salts. References hereinafter to a compound according to the invention include both 39-desmethoxyrapamycin and its pharmaceutically acceptable salts.

### DESCRIPTION OF THE INVENTION

The present invention relates to the use of a 39-desmethoxyrapamycin analogue in medicine, in particular in the treatment of cancer, B-cell malignancies, the induction or maintenance of immunosuppression, the treatment of transplantation rejection, graft vs. host disease, autoimmune disorders, neurodegenerative conditions, diseases of inflammation, vascular disease and fibrotic diseases, the stimulation of neuronal regeneration, the treatment of neurological diseases or neurodegenerative conditions or the treatment of fungal infections. Therefore, the present invention provides for the use of a 39-desmethoxyrapamycin analogue, or a pharmaceutically acceptable salt thereof, in the treatment of a medical condition resulting from neural injury or disease. In a specific embodiment, the present invention provides for the use of 39-desmethoxyrapamycin, or a pharmaceutically acceptable salt thereof, in the treatment of a medical condition resulting from neural injury or disease. In a further embodiment the present invention provides for the use of a 39-desmethoxyrapamycin analogue that additionally differs from rapamycin at one or more of positions 9, 16 or 27 in the treatment of a medical condition resulting from neural injury or disease.

The present invention also provides for the use of a 39-desmethoxyrapamycin analogue, i.e. a rapamycin analogue with increased blood-brain barrier permeability, or a pharmaceutically acceptable salt thereof, in the treatment of medical conditions affecting the central nervous which require the medicament to cross the blood-brain barrier i.e. medical conditions where the blood-brain barrier impedes the delivery of the compound. In a specific embodiment, the present invention provides for the use of 39-desmethoxyrapamycin, or a pharmaceutically acceptable salt thereof, in the treatment of medical conditions affecting the central nervous system where the blood-brain barrier impedes the delivery of the compound. In a further embodiment, the present invention provides for the use of a 39-desmethoxyrapamycin analogue that additionally differs from rapamycin at one or more of positions 9, 16 or 27, in the treatment of medical conditions affecting the central nervous system where the blood brain barrier impedes the delivery of the compound.

In a particular embodiment this invention relates to the use of a 39-desmethoxyrapamycin analogue for the treatment of cancer and B-cell malignancies. In a further embodiment this invention relates to the use of 39-desmethoxyrapamycin for the treatment of cancer and B-cell malignancies. In a further embodiment, the present invention relates to the use of a 39-desmethoxyrapamycin analogue that additionally differs from rapamycin at one or more of positions 9, 16 or 27 for the treatment of cancer and B-cell malignancies. The present invention also specifically provides for the use of a 39-desmethoxyrapamycin analogue in the treatment of brain tumour(s). The present invention further provides for the use of 39-desmethoxyrapamycin the treatment of brain tumour(s). In a further embodiment the present invention provides for the use of a 39-desmethoxyrapamycin analogue that additionally differs from rapamycin at one or more of positions 9, 16 or 27 in the treatment of brain tumour(s). In particular, the present invention provides for the use of a 39-desmethoxyrapamycin analogue in the treatment of glioblastoma multiforme. In a specific embodiment the present invention provides for the use of 39-desmethoxyrapamycin in the treatment of glioblastoma multiforme. In a further embodiment, the present invention provides for the use of a 39-desmethoxyrapamycin analogue that additionally differs from rapamycin at one or more of positions 9, 16 or 27 in the treatment of glioblastoma multiforme.

The present invention also provides for the use of a 39-desmethoxyrapamycin analogue in the treatment of neurodegenerative conditions. In a further embodiment the present invention provides for the use of 39-desmethoxyrapamycin in the treatment of neurodegenerative conditions. In a further embodiment the present invention provides for the use of a 39-desmethoxyrapamycin analogue that additionally differs from rapamycin at one or more of positions 9, 16 or 27 in the treatment of neurodegenerative conditions. Particularly, the neurodegenerative condition may be selected from the group consisting of Alzheimer's disease, multiple sclerosis and Huntington's disease. Therefore, in one embodiment the present invention provides for the use of a 39-desmethoxyrapamycin analogue in the treatment of Alzheimer's disease. In a further embodiment the present invention provides for the use of 39-desmethoxyrapamycin in the treatment of Alzheimer's disease. In a further embodiment the present invention provides for the use of a 39-desmethoxyrapamycin analogue that additionally differs from rapamycin at one or more of positions 9, 16 or 27 in the treatment of Alzheimer's disease. In a further embodiment the present invention provides for the use of a 39-desmethoxyrapamycin analogue in the treatment of multiple sclerosis. In a further embodiment the present invention provides for the use of 39-desmethoxyrapamycin in the treatment of multiple sclerosis. In a further embodiment, the present invention provides for the use of a 39-desmethoxyrapamycin analogue that additionally differs from rapamycin at one or more of positions 9, 16 or 27 in the treatment of multiple sclerosis. In an alternative embodiment, the present invention provides for the use of a 39-desmethoxyrapamycin analogue in the treatment of Huntington's disease. In a further embodiment, the present invention provides for the use of 39-desmethoxyrapamycin in the treatment of Huntington's disease. In a further embodiment, the present invention provides for the use of a 39-desmethoxyrapamycin analogue that additionally differs from rapamycin at one or more of positions 9, 16 or 27 in the treatment of Huntington's disease.

In an alternative embodiment, the present invention provides a method for the treatment of cancer or B-cell malignancies, a method of induction or maintenance of immunosuppression, the stimulation of neuronal regeneration, a method for the treatment of fungal infections, transplantation rejection, graft vs. host disease, autoimmune disorders, neurodegenerative conditions, diseases of inflammation vascular disease or fibrotic diseases which comprises administering to a patient an effective amount of a 39-desmethoxyrapamycin analogue, in particular 39-desmethoxyrapamycin or a 39-desmethoxyrapamycin analogue that additionally differs from rapamycin at one or more of positions 9, 16 or 27. Specifically, the present invention provides a method of treatment of a medical condition resulting from neural injury or disease, comprising administering a 39-desmethoxyrapamycin analogue, or a pharmaceutically acceptable salt thereof. In particular embodiment the present invention provides a method of treatment of a medical condition resulting from neural injury or disease, comprising administering 39-desmethoxyrapamycin. In a further embodiment, the present invention provides a method of treatment of a medical condition resulting from neural injury or disease, comprising administering a 39-desmethoxyrapamycin analogue that additionally differs from rapamycin at one or more of positions 9, 16 or 27. The present invention also provides a method of treatment of medical conditions affecting the central nervous system wherein the blood-brain barrier impedes the delivery of the compound, by administering an effective amount of a 39-desmethoxyrapamycin analogue, i.e. a rapamycin analogue with increased blood-brain barrier permeability, or a pharmaceutically acceptable salt thereof. In a specific aspect the 39-desmethoxyrapamycin analogue is 39-desmethoxyrapamycin. In a further aspect the 39-desmethoxyrapamycin analogue is a 39-desmethoxyrapamycin analogue that additionally differs from rapamycin at one or more of positions 9, 16 or 27.

Preferably, the present invention provides a method of treatment of cancer or B-cell malignancies which comprises administering to a patient an effective amount of a 39-desmethoxyrapamycin analogue. In a further preferred embodiment the present invention provides a method of treatment of brain tumour(s) which comprises administering to a patient an effective amount of a 39-desmethoxyrapamycin analogue. In a specific aspect the 39-desmethoxyrapamycin analogue is 39-desmethoxyrapamycin. In a further aspect the 39-desmethoxyrapamycin analogue is a 39-desmethoxyrapamycin analogue that additionally differs from rapamycin at one or more of positions 9, 16 or 27. In a particular embodiment the present invention provides a method of treatment of glioblastoma multiforme which comprises administering to a patient an effective amount of a 39-desmethoxyrapamycin analogue. In a specific aspect the 39-desmethoxyrapamycin analogue is 39-desmethoxyrapamycin. In a further aspect the 39-desmethoxyrapamycin analogue is a 39-desmethoxyrapamycin analogue that additionally differs from rapamycin at one or more of positions 9, 16 or 27.

In a further preferred embodiment the present invention provides a method of treatment of a neurodegenerative condition which comprises administering to a patient an effective amount of a 39-desmethoxyrapamycin analogue. In a specific aspect the 39-desmethoxyrapamycin analogue is 39-desmethoxyrapamycin. In a further aspect the 39-desmethoxyrapamycin analogue is a 39-desmethoxyrapamycin analogue that additionally differs from rapamycin at one or more of positions 9, 16 or 27. Particularly, the neurodegenerative condition may be selected from the group consisting of Alzheimer's disease, multiple sclerosis and Huntington's disease. Therefore, in one embodiment the present invention provides a method of treatment of Alzheimer's disease which comprises administering to a patient an effective amount of a 39-desmethoxyrapamycin analogue. In a specific aspect the 39-desmethoxyrapamycin analogue is 39-desmethoxyrapamycin. In a further aspect the 39-desmethoxyrapamycin analogue is a 39-desmethoxyrapamycin analogue that additionally differs from rapamycin at one or more of positions 9, 16 or 27. In a further embodiment the present invention a method of treatment of multiple sclerosis which comprises administering to a patient an effective amount of a 39-desmethoxyrapamycin analogue. In a specific aspect the 39-desmethoxyrapamycin analogue is 39-desmethoxyrapamycin. In a further aspect the 39-desmethoxyrapamycin analogue is a 39-desmethoxyrapamycin analogue that additionally differs from rapamycin at one or more of positions 9, 16 or 27. In an alternative embodiment, the present invention provides a method of treatment of Huntington's disease which comprises administering to a patient an effective amount of a 39-desmethoxyrapamycin analogue. In a specific aspect the 39-desmethoxyrapamycin analogue is 39-desmethoxyrapamycin. In a further aspect the 39-desmethoxyrapamycin analogue is a 39-desmethoxyrapamycin analogue that additionally differs from rapamycin at one or more of positions 9, 16 or 27.

The present invention also provides the use of a 39-desmethoxyrapamycin analogue in the manufacture of a medicament for treatment of cancer or B-cell malignancies, for induction or maintenance of immunosuppression, for stimulation of neuronal regeneration, for the treatment of fungal infections, transplantation rejection, graft vs. host disease, autoimmune disorders, neurodegenerative conditions, diseases of inflammation vascular disease or fibrotic diseases. Specifically, the present invention provides for the use of a 39-desmethoxyrapamycin analogue, or a pharmaceutically acceptable salt thereof, in the preparation of a medicament for the treatment of a medical condition resulting from neural injury or disease. In a specific aspect the 39-desmethoxyrapamycin analogue is 39-desmethoxyrapamycin. In a further aspect the 39-desmethoxyrapamycin analogue is a 39-desmethoxyrapamycin analogue that additionally differs from rapamycin at one or more of positions 9, 16 or 27.

The present invention also provides for the use of a 39-desmethoxyrapamycin analogue, i.e. a rapamycin analogue with increased blood-brain barrier permeability, or a pharmaceutically acceptable salt thereof, in the preparation of a medicament for the treatment of medical conditions affecting the central nervous system where the blood-brain barrier impedes the delivery of the compound. In a specific aspect the 39-desmethoxyrapamycin analogue is 39-desmethoxyrapamycin. In a further aspect the 39-desmethoxyrapamycin analogue is a 39-desmethoxyrapamycin analogue that additionally differs from rapamycin at one or more of positions 9, 16 or 27.

The present invention also specifically provides for the use of a 39-desmethoxyrapamycin analogue in the manufacture of a medicament for the treatment of brain tumour(s). In a specific aspect the 39-desmethoxyrapamycin analogue is 39-desmethoxyrapamycin. In a further aspect the 39-desmethoxyrapamycin analogue is a 39-desmethoxyrapamycin analogue that additionally differs from rapamycin at one or more of positions 9, 16 or 27. In a particular embodiment the present invention specifically provides for the use of a 39-desmethoxyrapamycin analogue in the manufacture of a medicament for the treatment of glioblastoma multiforme. In a specific aspect the 39-desmethoxyrapamycin analogue is 39-desmethoxyrapamycin. In a further aspect the 39-desmethoxyrapamycin analogue is a 39-desmethoxyrapamycin analogue that additionally differs from rapamycin at one or more of positions 9, 16 or 27.

The present invention also specifically provides for the use of a 39-desmethoxyrapamycin analogue in the manufacture of a medicament for the treatment of neurodegenerative conditions. In a specific aspect the 39-desmethoxyrapamycin analogue is 39-desmethoxyrapamycin. In a further aspect the 39-desmethoxyrapamycin analogue is a 39-desmethoxyrapamycin analogue that additionally differs from rapamycin at one or more of positions 9, 16 or 27. Particularly, the neurodegenerative condition may be selected from the group consisting of Alzheimer's disease, multiple sclerosis and Huntington's disease. Therefore, in one embodiment the present invention provides for the use of a 39-desmethoxyrapamycin analogue in the manufacture of a medicament for the treatment of Alzheimer's disease. In a specific aspect the 39-desmethoxyrapamycin analogue is 39-desmethoxyrapamycin. In a further aspect the 39-desmethoxyrapamycin analogue is a 39-desmethoxyrapamycin analogue that additionally differs from rapamycin at one or more of positions 9, 16 or 27. In a further embodiment the present invention provides for the use of a 39-desmethoxyrapamycin analogue in the manufacture of a medicament for the treatment of multiple sclerosis. In a specific aspect the 39-desmethoxyrapamycin analogue is 39-desmethoxyrapamycin. In a further aspect the 39-desmethoxyrapamycin analogue is a 39-desmethoxyrapamycin analogue that additionally differs from rapamycin at one or more of positions 9, 16 or 27. In an alternative embodiment, the present invention provides for the use of a 39-desmethoxyrapamycin analogue in the manufacture of a medicament for the treatment of Huntington's disease. In a specific aspect the 39-desmethoxyrapamycin analogue is 39-desmethoxyrapamycin. In a further aspect the 39-desmethoxyrapamycin analogue is a 39-desmethoxyrapamycin analogue that additionally differs from rapamycin at one or more of positions 9, 16 or 27.

39-Desmethoxyrapamycin analogues are close structural analogues of rapamycin that are made using the methods described in WO 04/007709. However they show a different spectrum of activity to rapamycin, for example as shown by the COMPARE analysis of the NCI 60 cell line panel for 39-desmethoxyrapamycin and related analogues (see table 1 below). The COMPARE analysis uses a Pearson analysis to compare the activity of two compounds on the NCI 60-cell line panel and produces a correlation coefficient which indicates how similar the two compounds spectra of activity are and this may indicate how related their mechanism's of action are. As a specific example, the Pearson coefficient for rapamycin and 39-desmethoxyrapamycin is 0.614, this should be compared to the Pearson coefficient between rapamycin and CCI-779 (a 40-hydroxy ester derivative of rapamycin) which is 0.86 (Dancey, 2002). Therefore, it can be seen that 39-desmethoxyrapamycin analogues have a different spectrum of activity compared to rapamycin.

TABLE 1

| | Pearson |
|---|---|
| | Coefficient vs. |
| Compound | rapamycin |
| 16-O-desmethyl-27-O-desmethyl-39- | 0.435 |
| desmethoxyrapamycin | |
| 27-O-desmethyl-39-desmethoxyrapamycin | 0.261 |
| 39-desmethoxyrapamycin | 0.614 |
| 27-desmethoxy-39-desmethoxy rapamycin | 0.313 |
| CC1-779 | 0.86 |

Multi-Drug Resistance (MDR) is a significant problem in the treatment of cancer and B-cell malignancies. It is the principle reason behind the development of drug resistance in many cancers (Persidis A, 1999). Drugs which worked initially become totally ineffective after a period of time. MDR is associated with increased level of adenosine triphosphate binding cassette transporters (ABC transporters), in particular an increase in the expression of the MDR1 gene which encodes for P-glycoprotein (P-gp) or the MRP1 gene which encodes MRP1. The level of MDR1 gene expression varies widely across different cancer-derived cell lines, in some cell lines it is undetectable, whereas in others may show up to a 10 or 100-fold increased expression relative to standard controls.

Some of the indicated difference in the spectrum of activity between rapamycin and 39-desmethoxyrapamycin may be explained because 39-desmethoxyrapamycin analogues are not a substrate for P-gp. 39-Desmethoxyrapamycin analogues have a decreased efflux from Caco-2 cells compared to rapamycin and 39-desmethoxyrapamycin was shown not to be a substrate for P-gp in an in vitro P-gp substrate assay (see examples herein).

Therefore, a further aspect of the invention provides for the use of a 39-desmethoxyrapamycin analogue in the treatment of a cancer or B-cell malignancy resistant to one or more existing anticancer agent(s) i.e. MDR cancers or B-cell malignancies. In a specific aspect the present invention provides for the use of 39-desmethoxyrapamycin in the treatment of P-gp-expressing cancers or B-cell malignancies. In a yet more preferred embodiment the present invention provides for the use of 39-desmethoxyrapamycin in the treatment of high P-gp expressing cancers or B-cell malignancies. Particularly, high P-gp expressing cancers or B-cell malignancies may have 2-fold, 5-fold, 10-fold, 20-fold, 25-fold, 50-fold or 100-fold increased expression relative to control levels. In a specific aspect of the above uses the 39-desmethoxyrapamycin analogue is 39-desmethoxyrapamycin. In a further aspect of the above uses the 39-desmethoxyrapamycin analogue is a 39-desmethoxyrapamycin analogue that additionally differs from rapamycin at one or more of positions 9, 16 or 27. Suitable controls are cells which do not express P-gp, which have a low expression level of P-gp or which have low MDR function, a person of skill in the art is aware of or can identify such cell lines; by way of example (but without limitation) suitable cell lines include: MDA435/LCC6, SBC-3/CDDP, MCF7, NCI-H23, NCI-H522, A549/ATCC, EKVX, NCI-H226, NCI-H322M, NCI-H460, HOP-18, HOP-92, LXFL 529, DMS 114, DMS 273, HT29, HCC-2998, HCT-116, COLO 205, KM12, KM20L2, MDA-MB-231/ATCC, MDA-MB-435, MDA-N, BT-549, T-47D, OVCAR-3, OVCAR-4, OVCAR-5, OVCAR-8, IGROV1, SK-OV-3, K-562, MOLT-4, HL-60(TB), RPMI-8226, SR, SN12C, RXF-631, 786-0, TK-10, LOX IMVI, MALME-3M, SK-MEL-2, SK-MEL-5, SK-MEL-28, M14, UACC-62, UACC-257, PC-3, DU-145, SNB-19, SNB-75, SNB-78, U251, SF-268, SF-539, XF 498.

In an alternative aspect the present invention provides for the use of a 39-desmethoxyrapamycin analogue in the preparation of a medicament for use in the treatment of

MDR cancers or B-cell malignancies. In a specific aspect the present invention provides for the use of a 39-desmethoxyrapamycin analogue in the preparation of a medicament for use in the treatment of P-gp-expressing cancers or B-cell malignancies. In a yet more preferred embodiment the present invention provides for the use of a 39-desmethoxyrapamycin analogue in the preparation of a medicament for use in the treatment of high P-gp expressing cancers or B-cell malignancies. Particularly, high P-gp expressing cancers or B-cell malignancies may have 2-fold, 5-fold, 10-fold, 20-fold, 25-fold, 50-fold or 100-fold increased expression relative to control levels. In a specific aspect the 39-desmethoxyrapamycin analogue is 39-desmethoxyrapamycin. In a further aspect the 39-desmethoxyrapamycin analogue is a 39-desmethoxyrapamycin analogue that additionally differs from rapamycin at one or more of positions 9, 16 or 27. Suitable controls are described above.

Methods for determining the expression level of P-gp in a sample are discussed further herein.

Therefore, in a further aspect the present invention provides a method for the treatment of P-gp-expressing-cancers or B-cell malignancies comprising administering a therapeutically effective amount of a 39-desmethoxyrapamycin analogue. In a specific aspect the 39-desmethoxyrapamycin analogue is 39-desmethoxyrapamycin. In a further aspect the 39-desmethoxyrapamycin analogue is a 39-desmethoxyrapamycin analogue that additionally differs from rapamycin at one or more of positions 9, 16 or 27. The expression level of P-glycoprotein (P-gp) in a particular cancer type may be determined by a person of skill in the art using techniques including but not limited to real time RT-PCR (Szakács et al, 2004; Stein et al, 2002; Langmann et al; 2003), by immunohistochemistry (Stein at al, 2002) or using microarrays (Lee et al, 2003), these methods are provided as examples only, other suitable methods will occur to a person of skill in the art.

39-Desmethoxyrapamycin shows increased metabolic stability compared to rapamycin as shown herein in the examples. A number of papers have previously identified the 39-methoxy group on rapamycin as being a major site of metabolic attack to convert rapamycin to 39-O-desmethylrapamycin (Trepanier et al, 1998). The major metabolites of rapamycin have significantly decreased activity when compared to the parent compound (Gallant-Haidner et al, 2000, Trepanier et al, 1998). In contrast, 39-desmethoxyrapamycin no longer has available the most significant sites of metabolic attack, which results in an increased stability of the compounds (see examples). Coupled with the higher or equivalent potency of 39-desmethoxyrapamycin to the rapamycin parent compound this provides a longer half-life for the compound of the invention. This is a further surprising advantage of 39-desmethoxyrapamycin over rapamycin.

The properties of 39-desmethoxyrapamycin described above (that it is not a substrate for P-gp, has increased metabolic stability and decreased efflux from cells via P-gp) indicate that 39-desmethoxyrapamycin has improved bioavailability compared to its parent compound rapamycin. Therefore, the present invention provides for the use of 39-desmethoxyrapamycin, a rapamycin analogue with improved metabolic stability, improved cell membrane permeability and a distinct cancer cell inhibitory profile, in medicine, particularly in the treatment of cancer or B-cell malignancies.

The present invention also provides a pharmaceutical composition comprising a 39-desmethoxyrapamycin analogue, or a pharmaceutically acceptable salt thereof, together with a pharmaceutically acceptable carrier. In a specific aspect the present invention provides a pharmaceutical composition comprising 39-desmethoxyrapamycin. In a further aspect the present invention provides a pharmaceutical composition comprising a 39-desmethoxyrapamycin analogue that additionally differs from rapamycin at one or more of positions 9, 16 or 27. In a specific embodiment the present invention provides a pharmaceutical composition as described above that is specifically formulated for intravenous administration.

Rapamycin and related compounds that are or have been in clinical trials, such as CCI-779 and RAD001 have poor pharmacological profiles, including poor metabolic stability, poor permeability, high levels of efflux via P-gp and poor bioavailability. The present invention provides for the use of a 39-desmethoxyrapamycin analogue or a pharmaceutically acceptable salt thereof which has improved pharmaceutical properties compared to rapamycin.

A further surprising aspect of the present invention is that 39-desmethoxyrapamycin analogues display a strikingly different pharmacokinetic profile when compared to the existing rapamycin analogues. In particular, 39-desmethoxyrapamycin analogues show increased blood brain barrier permeability and thus higher exposure of these compounds is seen in the brain compared to related analogues for a given blood level.

This difference in pharmacokinetics is entirely unexpected and is not suggested anywhere in the prior art. A known disadvantage with currently available therapies for disorders including neurodegenerative conditions and brain tumours is the challenge of getting the drugs to the site of action (see Pardridge, 2005). This has also been reported to be a problem with existing rapamycin analogs when used in therapy, in particular a study investigating the efficacy of CCI-779 in the treatment of glioblastoma multiforme concluded that although systemic concentrations were adequate, the blood-brain barrier had acted as a barrier for delivery of the drug to the tumour (Chang, 2005) The present invention therefore discloses for the first time a rapamycin analogue with improved blood-brain barrier permeability and therefore significant utility for treating brain tumours and neurodegenerative conditions.

Preferred 39-desmethoxyrapamycin analogues for use in any of the aspects of the invention described above include those which additionally differ from rapamycin at any one of positions 9, 16 or 27, i.e. it is preferred that the 39-desmethoxyrapamycin analogue is not 39-desmethoxyrapamycin itself. Further preferred 39-desmethoxyrapamycin analogues include those wherein:
the 39-desmethoxyrapamycin analogue has a hydroxyl group at position 27, i.e. R₃ represents OH;
the 39-desmethoxyrapamycin analogue has a hydrogen at position 27, i.e. R₃ represents OH; or
the 39-desmethoxyrapamycin analogue has a hydroxyl group at position 16, i.e. R₂ represents OH.

A person of skill in the art will be able to determine the pharmacokinetics and bioavailability of a compound of the invention using in vivo and in vitro methods known to a person of skill in the art, including but not limited to those described below and in Gallant-Haidner et al, 2000 and Trepanier et al, 1998 and references therein. The bioavailability of a compound is determined by a number of factors, (e.g. water solubility, cell membrane permeability, the extent of protein binding and metabolism and stability) each of which may be determined by in vitro tests as described in the examples herein, it will be appreciated by a person of skill in the art that an improvement in one or more of these factors will lead to an improvement in the bioavailability of a compound. Alternatively, the bioavailability of 39-desmethoxyrapamycin or a pharmaceutically acceptable salt thereof may be measured using in vivo methods as described in more detail below, or in the examples herein.

In Vivo Assays

In vivo assays may also be used to measure the bioavailability of a compound such as 39-desmethoxyrapamcyin. Generally, said compound is administered to a test animal (e.g. mouse or rat) both intraperitoneally (i.p.) or intravenously (i.v.) and orally (p.o.) and blood samples are taken at regular intervals to examine how the plasma concentration of the drug varies over time. The time course of plasma concentration over time can be used to calculate the absolute bioavailability of the compound as a percentage using standard models. An example of a typical protocol is described below.

Mice are dosed with 3 mg/kg of 39-desmethoxyrapamycin i.v. or 10 mg/kg of 39-desmethoxyrapamycin p.o. Blood samples are taken at 5 min, 15 min, 1 h, 4 h and 24 h intervals, and the concentration of 39-desmethoxyrapamycin in the sample is determined via HPLC. The time-course of plasma or whole blood concentrations can then be used to derive key parameters such as the area under the plasma or blood concentration-time curve (AUC—which is directly proportional to the total amount of unchanged drug that reaches the systemic circulation), the maximum (peak) plasma or blood drug concentration, the time at which maximum plasma or blood drug concentration occurs (peak time), additional factors which are used in the accurate determination of bioavailability include: the compound's terminal half life, total body clearance, steady-state volume of distribution and F %. These parameters are then analysed by non-compartmental or compartmental methods to give a calculated percentage bioavailability, for an example of this type of method see Gallant-Haidner et al, 2000 and Trepanier et al, 1998, and references therein.

The efficacy of 39-desmethoxyrapamycin may be tested in in vivo models for neurodegenerative diseases which are described herein and which are known to a person of skill in the art. Such models include, but are not limited to, for Alzheimer's disease—animals that express human familial Alzheimer's disease (FAD) p-amyloid precursor (APP), animals that overexpress human wild-type APP, animals that overexpress p-amyloid 1-42(pA), animals that express FAD presenillin-1 (PS-1) (e. g. German and Eisch, 2004). For multiple sclerosis—the experimental autoimmune encephalomyelitis (EAE) model (see Bradl, 2003 and Example 7). For Parkinson's disease—the 1-methyl-4-phenyl-1,2,3,6-tetrahydropyridine (MPTP) model or the 6-hydroxydopamine (6-OHDA) model (see e.g. Emborg, 2004; Schober A. 2004). For Huntington's disease there are several models including the R6 lines model generated by the introduction of exon 1 of the human Huntington's disease (HD) gene carrying highly expanded CAG repeats into the mouse germ line (Sathasivam at al, 1999) and others (see Hersch and Ferrante, 2004).

The aforementioned compound of the invention or a formulation thereof may be administered by any conventional method for example but without limitation they may be administered parenterally, orally, topically (including buccal, sublingual or transdermal), via a medical device (e.g. a stent), by inhalation or via injection (subcutaneous or intramuscular). The treatment may consist of a single dose or a plurality of doses over a period of time.

Whilst it is possible for a 39-desmethoxyrapamycin analogue to be administered alone, it is preferable to present it as a pharmaceutical formulation, together with one or more acceptable carriers. The carrier(s) must be “acceptable” in the sense of being compatible with the compound of the invention and not deleterious to the recipients thereof. Examples of suitable carriers are described in more detail below.

A 39-desmethoxyrapamycin analogue may be administered alone or in combination with other therapeutic agents, co-administration of two (or more) agents allows for significantly lower doses of each to be used, thereby reducing the side effects seen. The increased metabolic stability of 39-desmethoxyrapamycin has an extra advantage over rapamycin in that it is less likely to cause drug-drug interactions when used in combination with drugs that are substrates of P450 enzymes as occurs with rapamycin (Lampen at al, 1998).

Therefore in one embodiment, a 39-desmethoxyrapamycin analogue is co-administered with another therapeutic agent for the induction or maintenance of immunosuppression, for the treatment of transplantation rejection, graft vs. host disease, autoimmune disorders or diseases of inflammation preferred agents include, but are not limited to, immunoregulatory agents e.g. azathioprine, corticosteroids, cyclophosphamide, cyclosporin A, FK506, Mycophenolate Mofetil, OKT-3 and ATG.

In a alternative embodiment, a 39-desmethoxyrapamycin analogue is co-administered with another therapeutic agent for the treatment of cancer or B-cell malignancies preferred agents include, but are not limited to, methotrexate, leukovorin, adriamycin, prenisone, bleomycin, cyclophosphamide, 5-fluorouracil, paclitaxel, docetaxel, vincristine, vinblastine, vinorelbine, doxorubicin, tamoxifen, toremifene, megestrol acetate, anastrozole, goserelin, anti-HER2 monoclonal antibody (e.g. Herceptin™), capecitabine, raloxifene hydrochloride, EGFR inhibitors (e.g. Iressa®, Tarceva™, Erbitux™), VEGF inhibitors (e.g. Avastin™), proteasome inhibitors (e.g. Velcade™), Glivec® or hsp90 inhibitors (e.g. 17-AAG). Additionally, 39-desmethoxyrapamycin may be administered in combination with other therapies including, but not limited to, radiotherapy or surgery.

In one embodiment, a 39-desmethoxyrapamycin analogue is co-administered with another therapeutic agent for the treatment of vascular disease, preferred agents include, but are not limited to, ACE inhibitors, angiotensin II receptor antagonists, fibric acid derivatives, HMG-CoA reductase inhibitors, beta adrenergic blocking agents, calcium channel blockers, antioxidants, anticoagulants and platelet inhibitors (e.g. Plavix™).

In one embodiment, a 39-desmethoxyrapamycin analogue is co-administered with another therapeutic agent for the stimulation of neuronal regeneration, preferred agents include, but are not limited to, neurotrophic factors e.g. nerve growth factor, glial derived growth factor, brain derived growth factor, ciliary neurotrophic factor and neurotrophin-3.

In one embodiment, a 39-desmethoxyrapamycin analogue is co-administered with another therapeutic agent for the treatment of fungal infections; preferred agents include, but are not limited to, amphotericin B, flucytosine, echinocandins (e.g. caspofungin, anidulafungin or micafungin), griseofulvin, an imidazole or a triazole antifungal agent (e.g. clotrimazole, miconazole, ketoconazole, econazole, butoconazole, oxiconazole, terconazole, itraconazole, fluconazole or voriconazole).

In one embodiment, a 39-desmethoxyrapamycin analogue is co-administered with another therapeutic agent for the treatment of Alzheimer's disease; preferred agents include, but are not limited to, cholinesterase inhibitors e.g. donepezil, rivastigmine, and galantamine; N-methyl-D-aspartate (NMDA) receptor antagonists, e.g, Memantine.

In one embodiment, a 39-desmethoxyrapamycin analogue is co-administered with another therapeutic agent for the treatment of multiple sclerosis; preferred agents include, but are not limited to, Interferon beta-1b, Interferon beta-1a, glatiramer, mitoxantrone, cyclophosphamide, corticosteroids (e.g. methylprednisolone, prednisone, dexamethasone).

By co-administration is included any means of delivering two or more therapeutic agents to the patient as part of the same treatment regime, as will be apparent to the skilled person. Whilst the two or more agents may be administered simultaneously in a single formulation this is not essential. The agents may administered in different formulations and at different times.

The formulations may conveniently be presented in unit dosage form and may be prepared by any of the methods well known in the art of pharmacy. Such methods include the step of bringing into association the active ingredient (compound of the invention) with the carrier which constitutes one or more accessory ingredients. In general the formulations are prepared by uniformly and intimately bringing into association the active ingredient with liquid carriers or finely divided solid carriers or both, and then, if necessary, shaping the product.

A 39-desmethoxyrapamycin analogue will normally be administered intravenously, orally or by any parenteral route, in the form of a pharmaceutical formulation comprising the active ingredient, optionally in the form of a non-toxic organic, or inorganic, acid, or base, addition salt, in a pharmaceutically acceptable dosage form. Depending upon the disorder and patient to be treated, as well as the route of administration, the compositions may be administered at varying doses.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability.

The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), vegetable oils, and suitable mixtures thereof.

For example, a 39-desmethoxyrapamycin analogue can be administered orally, buccally or sublingually in the form of tablets, capsules, ovules, elixirs, solutions or suspensions, which may contain flavouring or colouring agents, for immediate-, delayed- or controlled-release applications.

Solutions or suspensions of a 39-desmethoxyrapamycin analogue suitable for oral administration may also contain excipients e.g. N,N-dimethylacetamide, dispersants e.g. polysorbate 80, surfactants, and solubilisers, e.g. polyethylene glycol, Phosal 50 PG (which consists of phosphatidylcholine, soya-fatty acids, ethanol, mono/diglycerides, propylene glycol and ascorbyl palmitate).

Such tablets may contain excipients such as microcrystalline cellulose, lactose (e.g. lactose monohydrate or lactose anyhydrous), sodium citrate, calcium carbonate, dibasic calcium phosphate and glycine, butylated hydroxytoluene (E321), crospovidone, hypromellose, disintegrants such as starch (preferably corn, potato or tapioca starch), sodium starch glycollate, croscarmellose sodium, and certain complex silicates, and granulation binders such as polyvinylpyrrolidone, hydroxypropylmethylcellulose (HPMC), hydroxy-propylcellulose (HPC), macrogol 8000, sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the compounds of the invention may be combined with various sweetening or flavouring agents, colouring matter or dyes, with emulsifying and/or suspending agents and with diluents such as water, ethanol, propylene glycol and glycerin, and combinations thereof.

A tablet may be made by compression or moulding, optionally with one or more accessory ingredients. Compressed tablets may be prepared by compressing in a suitable machine the active ingredient in a free-flowing form such as a powder or granules, optionally mixed with a binder (e.g. povidone, gelatin, hydroxypropylmethyl cellulose), lubricant, inert diluent, preservative, disintegrant (e.g. sodium starch glycolate, cross-linked povidone, cross-linked sodium carboxymethyl cellulose), surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethylcellulose in varying proportions to provide desired release profile.

Formulations in accordance with the present invention suitable for oral administration may be presented as discrete units such as capsules, cachets or tablets, each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Formulations suitable for topical administration in the mouth include lozenges comprising the active ingredient in a flavoured basis, usually sucrose and acacia or tragacanth; pastilles comprising the active ingredient in an inert basis such as gelatin and glycerin, or sucrose and acacia; and mouth-washes comprising the active ingredient in a suitable liquid carrier,

It should be understood that in addition to the ingredients particularly mentioned above the formulations of this invention may include other agents conventional in the art having regard to the type of formulation in question, for example those suitable for oral administration may include flavouring agents.

Pharmaceutical compositions adapted for topical administration may be formulated as ointments, creams, suspensions, lotions, powders, solutions, pastes, gels, impregnated dressings, sprays, aerosols or oils, transdermal devices, dusting powders, and the like. These compositions may be prepared via conventional methods containing the active agent. Thus, they may also comprise compatible conventional carriers and additives, such as preservatives, solvents to assist drug penetration, emollient in creams or ointments and ethanol or oleyl alcohol for lotions. Such carriers may be present as from about 1% up to about 98% of the composition. More usually they will form up to about 80% of the composition. As an illustration only, a cream or ointment is prepared by mixing sufficient quantities of hydrophilic material and water, containing from about 5-10% by weight of the compound, in sufficient quantities to produce a cream or ointment having the desired consistency.

Pharmaceutical compositions adapted for transdermal administration may be presented as discrete patches intended to remain in intimate contact with the epidermis of the recipient for a prolonged period of time. For example, the active agent may be delivered from the patch by iontophoresis.

For applications to external tissues, for example the mouth and skin, the compositions are preferably applied as a topical ointment or cream. When formulated in an ointment, the active agent may be employed with either a paraffinic or a water-miscible ointment base.

Alternatively, the active agent may be formulated in a cream with an oil-in-water cream base or a water-in-oil base.

For parenteral administration, fluid unit dosage forms are prepared utilizing the active ingredient and a sterile vehicle, for example but without limitation water, alcohols, polyols, glycerine and vegetable oils, water being preferred. The active ingredient, depending on the vehicle and concentration used, can be either suspended or dissolved in the vehicle. In preparing solutions the active ingredient can be dissolved in water for injection and filter sterilised before filling into a suitable vial or ampoule and sealing.

Advantageously, agents such as local anaesthetics, preservatives and buffering agents can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use.

Parenteral suspensions are prepared in substantially the same manner as solutions, except that the active ingredient is suspended in the vehicle instead of being dissolved and sterilization cannot be accomplished by filtration. The active ingredient can be sterilised by exposure to ethylene oxide before suspending in the sterile vehicle. Advantageously, a surfactant or wetting agent is included in the composition to facilitate uniform distribution of the active ingredient.

A 39-desmethoxyrapamycin analogue may also be administered using medical devices known in the art. For example, in one embodiment, a pharmaceutical composition of the invention can be administered with a needleless hypodermic injection device, such as the devices disclosed in U.S. Pat. No. 5,399,163; U.S. Pat. No. 5,383,851; U.S. Pat. No. 5,312,335; U.S. Pat. No. 5,064,413; U.S. Pat. No. 4,941,880; U.S. Pat. No. 4,790,824; or U.S. Pat. No.4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. In a specific embodiment a 39-desmethoxyrapamycin analogue may be administered using a drug-eluting stent, for example one corresponding to those described in WO 01/87263 and related publications or those described by Perin (Perin, E C, 2005). Many other such implants, delivery systems, and modules are known to those skilled in the art.

The dosage to be administered of a compound of the invention will vary according to the particular compound, the disease involved, the subject, and the nature and severity of the disease and the physical condition of the subject, and the selected route of administration. The appropriate dosage can be readily determined by a person skilled in the art.

The compositions may contain from 0.1% by weight, preferably from 5-60%, more preferably from 10-30% by weight, of a compound of invention, depending on the method of administration.

It will be recognized by one of skill in the art that the optimal quantity and spacing of individual dosages of a compound of the invention will be determined by the nature and extent of the condition being treated, the form, route and site of administration, and the age and condition of the particular subject being treated, and that a physician will ultimately determine appropriate dosages to be used. This dosage may be repeated as often as appropriate. If side effects develop the amount and/or frequency of the dosage can be altered or reduced, in accordance with normal clinical practice.

### BRIEF DESCRIPTION OF THE DRAWINGS

FIG. 1: shows the structure of rapamycin

FIG. 2: shows the fragmentation pathway for 39-desmethoxyrapamycin FIG. 3: shows western blots summarisng the mTOR inhibitory activity of 39-desmethoxyrapamycin and rapamycin.

FIG. 4: the % T/C values at all test concentrations for paclitaxel (A and C) and 39-desmethoxyrapamycin (B and D) in normal (A and B) or high P-gp expressing (C and D) cell lines.

FIG. 5: A—shows the total Area under the Curve (AUC) from 0-24 h for brain tissue or blood samples after a single i.v. or p.o. administration of rapamycin and 39-desmethoxyrapamycin.

B—shows the level of 39-desmethoxyrapamycin and rapamycin detected in the brain tissue over time after a single i.v. administration.

FIG. 6: A—shows disease progression in the EAE model under the prophylactic regime.

Values given are the median from the vehicle or treated group.

B—shows disease progression in the EAE model under the therapeutic regime. Values given are the median from the vehicle or treated group.

FIG. 7: the graph indicates the relative % survival of mice after induction of glioma by stereotaxic injection of U87-MG cells. Filled diamonds represent the untreated group, filled squares represent the vehicle treated group and open circles represent the 39-desmethoxyrapamycin treated group.

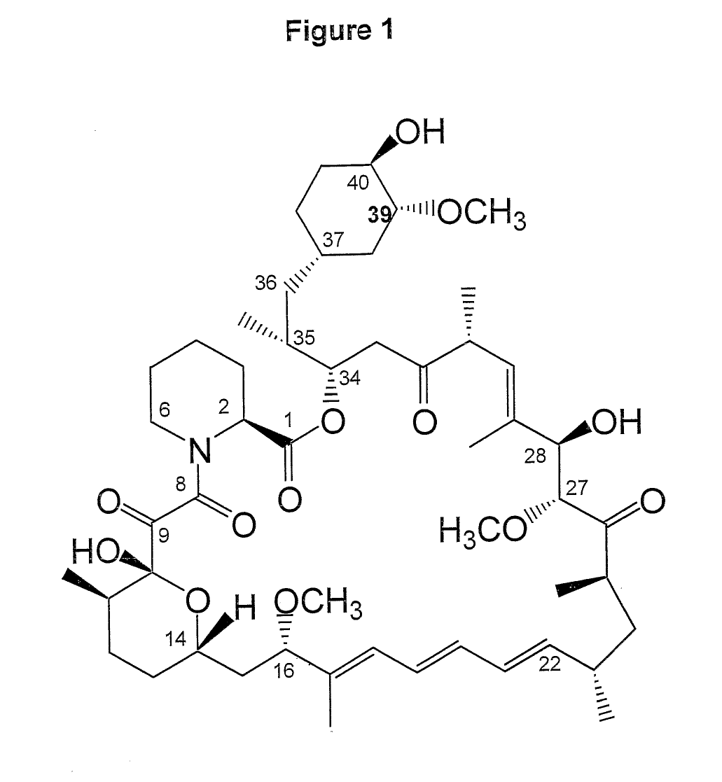

### EXAMPLES

### Materials & Methods

### Materials

Unless otherwise indicated, all reagents used in the examples below were obtained from commercial sources.

### Culture

S. hygroscopicus MG2-10 [IJMNOQLhis] (WO 04/007709; Gregory et al., 2004) was maintained on medium 1 agar plates (see below) at 28° C. Spore stocks were prepared after growth on medium 1, preserved in 20% w/v glycerol:10% w/v lactose in distilled water and stored at −80° C. Vegetative cultures were prepared by inoculating 0.1 mL of frozen stock into 50 mL medium 2 (see below) in 250 mL flask. The culture was incubated for 36 to 48 hours at 28° C., 300 rpm.

### Production Method:

Vegetative cultures were inoculated at 2.5-5% v/v into medium 3. Cultivation was carried out for 6-7 days, 26° C., 300 rpm.

### Feeding Procedure:

The feeding/addition of cyclohexane carboxylic acid was carried out 24-48 hours after inoculation and was fed at 1-2 mM final concentration unless stated otherwise.

Medium 1:

| component | Source | Catalogue # | Per L | |
|---|---|---|---|---|
| Corn steep powder | Sigma | C-8160 | 2.5 | g |
| Yeast extract | Difco | 0127-17 | 3 | g |
| Calcium carbonate | Sigma | C5929 | 3 | g |
| Iron sulphate | Sigma | F8633 | 0.3 | g |
| BACTO agar | | | 20 | g |
| Wheat starch | Sigma | S2760 | 10 | g |
| Water to | | | 1 | L |

The media was then sterilised by autoclaving 121° C., 20 min.

Medium 2: RapV7 Seed medium

| Component | Per L | |
|---|---|---|
| Toasted Nutrisoy (ADM Ingredients Ltd) | 5 | g |
| Avedex W80 dextrin (Deymer Ingredients Ltd) | 35 | g |
| Corn Steep Solids (Sigma) | 4 | g |
| Glucose | 10 | g |
| (NH₄)₂SO₄ | 2 | g |
| Lactic acid (80%) | 1.6 | mL |
| CaCO₃ (Caltec) | 7 | g |

Adjust pH to 7.5 with 1 M NaOH.
The media was then sterilised by autoclaving 121° C., 20 min.

After sterilisation 0.16 mL of 40% glucose is added to each 7 mL of media.

Medium 3: MD6 medium (Fermentation medium)

| Component | Per L | |
|---|---|---|
| Toasted Nutrisoy (ADM Ingredients Ltd) | 30 | g |
| Corn starch (Sigma) | 30 | g |
| Avedex W80 dextrin (Deymer Ingredients Ltd) | 19 | g |
| Yeast (Allinson) | 3 | g |
| Corn Steep Solids (Sigma) | 1 | g |
| KH₂PO₄ | 2.5 | g |
| K₂HPO₄ | 2.5 | g |
| (NH₄)₂SO₄ | 10 | g |
| NaCl | 5 | g |
| CaCO₃ (Caltec) | 10 | g |
| MnCl₂•4H₂O | 10 | mg |
| MgSO₄•7H₂O | 2.5 | mg |
| FeSO₄•7H₂O | 120 | mg |
| ZnSO₄•7H₂O | 50 | mg |
| MES (2-morpholinoethane sulphuric acid monohydrate) | 21.2 | g |

pH is corrected to 6.0 with 1 M NaOH
Before sterilization 0.4 mL of Sigma α-amylase (BAN 250) was added to 1 L of medium.
Medium was sterilised for 20 min at 121° C.
After sterilisation 0.35 mL of sterile 40% fructose and 0.10 mL of L-lysine (140 mg/mL in water, filter-sterilsed) was added to each 7 mL.

Medium 4: Rap V7a Seed medium

| Component | Per L | |
|---|---|---|
| Toasted Nutrisoy (ADM Ingredients Ltd) | 5 | g |
| Avedex W80 dextrin (Deymer Ingredients Ltd) | 35 | g |
| Corn Steep Solids (Sigma) | 4 | g |
| (NH₄)₂SO₄ | 2 | g |
| Lactic acid (80%) | 1.6 | mL |
| CaCO₃ (Caltec) | 7 | g |

Adjust pH to 7.5 with 1 M NaOH.
The media was then sterilised by autoclaving 121° C., 20 min.

Medium 5: MD6/5-1 medium (Fermentation medium)

| Component | Per L | |
|---|---|---|
| Toasted Nutrisoy (ADM Ingredients Ltd) | 15 | g |
| Avedex W80 dextrin (Deymer Ingredients Ltd) | 50 | g |
| Yeast (Allinson) | 3 | g |
| Corn Steep Solids (Sigma) | 1 | g |
| KH₂PO₄ | 2.5 | g |
| K₂HPO₄ | 2.5 | g |
| (NH₄)₂SO₄ | 10 | g |
| NaCl | 13 | g |
| CaCO₃ (Caltec) | 10 | g |
| MnCl₂•4H₂O | 3.5 | mg |
| MgSO₄•7H₂O | 15 | mg |
| FeSO₄•7H₂O | 150 | mg |
| ZnSO₄•7H₂O | 60 | mg |
| SAG 471 | 0.1 | ml |

Medium was sterilised for 30 min at 121° C.
After sterilisation 15 g of Fructose per L was added.
After 48 h 0.5 g/L of L-lysine was added.

### Analytical Methods

### Method A

Injection volume: 0.005-0.1 mL (as required depending on sensitivity). HPLC was performed on Agilent “Spherisorb” “Rapid Resolution” cartridges SB C8, 3 micron, 30 mm×2.1 mm, running a mobile phase of:

Mobile phase A: 0.01% Formic acid in pure water

Mobile phase B: 0.01% Formic acid in Acetonitrile

Flow rate: 1 mL/minute.

Linear gradient was used from 5% B at 0 min to 95% B at 2.5 min holding at 95% B until 4 min returning to 5% B until next cycle. Detection was by UV absorbance at 254 nm and/or by mass spectrometry electrospray ionisation (positive or negative) using a Micromasss Quattro-Micro instrument.

### Method B

Injection volume: 0.02 mL. HPLC was performed on 3 micron BDS C18 Hypersil

(ThermoHypersil-Keystone Ltd) column, 150×4.6 mm, maintained at 50° C., running a mobile phase of:
Mobile phase A: Acetonitrile (100 mL), trifluoracetic acid (1 mL), 1 M ammonium acetate (10 mL) made up to 1 L with deionised water.
Mobile phase B: Deionised water (100 mL), trifluoracetic acid (1 mL), 1M ammonium acetate (10 mL) made up to 1 L with acetonitrile.
Flow rate: 1 mL/minute.

A linear gradient from 55% B-95% B was used over 10 minutes, followed by 2 minutes at 95% B, 0.5 minutes to 55% B and a further 2.5 minutes at 55% B. Compound detection was by UV absorbance at 280 nm.

### Method C

The HPLC system comprised an Agilent HP1100 and was performed on 3 micron BDS C18 Hypersil (ThermoHypersil-Keystone Ltd) column, 150×4.6 mm, maintained at 40° C., running a mobile phase of:

Mobile phase A: deionised water.

Mobile phase B: acetonitrile.

Flow rate: 1 mL/minute.

This system was coupled to a Bruker Daltonics Esquire3000 electrospray mass spectrometer. Positive negative switching was used over a scan range of 500 to 1000 Dalton.

A linear gradient from 55% B-95% B was used over 10 minutes, followed by 2 minutes at 95% B, 0.5 minutes to 55% B and a further 2.5 minutes at 55% B.

### In Vitro Bioassay for Anticancer Activity

In vitro evaluation of compounds for anticancer activity in a panel of 12 human tumour cell lines in a monolayer proliferation assay were carried out at the Oncotest Testing Facility, Institute for Experimental Oncology, Oncotest GmbH, Freiburg. The characteristics of the 12 selected cell lines is summarised in Table 2.

TABLE 2
Test cell lines

| # | Cell line | Characteristics |
|---|---|---|
| 1 | MCF-7 | Breast, NCl standard |
| 2 | MDA-MB-231 | Breast - PTEN positive, resistant to 17-AAG |
| 3 | MDA-MB-468 | Breast - PTEN negative, resistant to 17-AAG |
| 4 | NCl-H460 | Lung, NCl standard |
| 5 | SF-268 | CNS, NCl standard |
| 6 | OVCAR-3 | Ovarian - p85 mutated. AKT amplified. |
| 7 | A498 | Renal, high MDR expression, |
| 8 | GXF 251L | Gastric |
| 9 | MEXF 394NL | Melanoma |
| 10 | UXF 1138L | Uterus |
| 11 | LNCAP | Prostate - PTEN negative |
| 12 | DU145 | Prostate - PTEN positive |

The Oncotest cell lines were established from human tumor xenografts as described by Roth at al. 1999. The origin of the donor xenografts was described by Fiebig at al. 1992. Other cell lines were either obtained from the NCI (H460, SF-268, OVCAR-3, DU145, MDA-MB-231, MDA-MB-468) or purchased from DSMZ, Braunschweig, Germany (LNCAP).

All cell lines, unless otherwise specified, are grown at 37° C. in a humidified atmosphere (95% air, 5% CO₂) in a ‘ready-mix’ medium containing RPMI 1640 medium, 10% fetal calf serum, and 0.1 mg/mL gentamicin (PAA, Cölbe, Germany).

Monolayer Assay—Protocol 1

A modified propidium iodide assay was used to assess the effects of the test compound(s) on the growth of twelve human tumor cell lines (Dengler et al, 1995).

Briefly, cells were harvested from exponential phase cultures by trypsinization, counted and plated in 96 well flat-bottomed microtitre plates at a cell density dependent on the cell line (5-10.000 viable cells/well). After 24 h recovery to allow the cells to resume exponential growth, 0.01 mL of culture medium (6 control wells per plate) or culture medium containing 39-desmethoxyrapamycin were added to the wells. Each concentration was plated in triplicate. 39-Desmethoxyrapamycin was applied in two concentrations (0.001 mM and 0.01 mM). Following 4 days of continuous incubation, cell culture medium with or without 39-desmethoxyrapamycin was replaced by 0.2 mL of an aqueous propidium iodide (PI) solution (7 mg/L). To measure the proportion of living cells, cells were permeabilized by freezing the plates. After thawing the plates, fluorescence was measured using the Cytofluor 4000 microplate reader (excitation 530 nm, emission 620 nm), giving a direct relationship to the total number of viable cells.

Growth inhibition was expressed as treated/control×100 (% TIC). For active compounds, IC₅₀ & IC₇₀ values were estimated by plotting compound concentration versus cell viability.

### Monolayer Assay—Protocol 2:

The human tumor cell lines of the National Cancer Institute (NCI) cancer screening panel were grown in RPMI 1640 medium containing 5% fetal bovine serum and 2 mM L-glutamine (Boyd and Paull, 1995). Cells were inoculated into 96 well microtiter plates in 0.1 mL at plating densities ranging from 5,000 to 40,000 cells/well depending on the doubling time of individual cell lines. After cell inoculation, the microtiter plates were incubated at 37° C., 5% CO₂, 95% air and 100% relative humidity for 24 h prior to addition of experimental drugs.

After 24 h, two plates of each cell line were fixed in situ with trichloroacetic acid (TCA), to represent a measurement of the cell population for each cell line at the time of drug addition (Tz). Experimental drugs were solubilized in dimethyl sulfoxide at 400-fold the desired final maximum test concentration and stored frozen prior to use. At the time of drug addition, an aliquot of frozen concentrate was thawed and diluted to twice the desired final maximum test concentration with complete medium containing 0.05 mg/mL gentamicin. Additional four, 10-fold or ½ log serial dilutions were made to provide a total of five drug concentrations plus control. Aliquots of 0.1 mL of these different drug dilutions were added to the appropriate microtiter wells already containing 0.1 mL of medium, resulting in the required final drug concentrations.

Following drug addition, the plates were incubated for an additional 48 h at 37° C., 5% CO₂, 95% air, and 100% relative humidity. For adherent cells, the assay was terminated by the addition of cold TCA. Cells were fixed in situ by the gentle addition of 0.05 mL of cold 50% (w/v) TCA (final concentration, 10% TCA) and incubated for 60 minutes at 4° C. The supernatant was discarded, and the plates were washed five times with tap water and air dried. Sulforhodamine B (SRB) solution (0.1 mL) at 0.4% (w/v) in 1% acetic acid was added to each well, and plates were incubated for 10 minutes at room temperature. After staining, unbound dye was removed by washing five times with 1% acetic acid and the plates were air dried. Bound stain was subsequently solubilized with 10 mM trizma base, and the absorbance was read on an automated plate reader at a wavelength of 515 nm. For suspension cells, the methodology was the same except that the assay was terminated by fixing settled cells at the bottom of the wells by gently adding 0.05 mL of 80% TCA (final concentration, 16% TCA). Using the seven absorbance measurements [time zero, (Tz), control growth, (C), and test growth in the presence of drug at the five concentration levels (Ti)], the percentage growth was calculated at each of the drug concentrations levels. Percentage growth inhibition was calculated as:

[(Ti−Tz)/(C−Tz)]×100 for concentrations where Ti≧Tz

[(Ti−Tz)/Tz]×100 for concentrations where Ti≦Tz.

Three dose response parameters were calculated for each experimental agent. Growth inhibition of 50% (GI50) was calculated from [(Ti−Tz)/(C−Tz)]×100=50, which is the drug concentration resulting in a 50% reduction in the net protein increase (as measured by SRB staining) in control cells during the drug incubation. The drug concentration resulting in total growth inhibition (TGI) was calculated from Ti=Tz. The LC50 (concentration of drug resulting in a 50% reduction in the measured protein at the end of the drug treatment as compared to that at the beginning) indicating a net loss of cells following treatment was calculated from [(Ti−Tz)/Tz]×100=−50.

Multi-drug resistant cell lines within the 60 cell line panel were identified by the NCI as high P-gp containing cell lines as identified by rhodamine B efflux (Lee et al., 1994) and by PCR detection of mRNA of mdr-1 (Alvarez et al., 1995).

### Pharmacokinetic Analysis—Protocol

The test compounds were prepared in a vehicle consisting of 4% Ethanol, 5% Tween-20, 5% polyethyleneglycol 400 in 0.15M NaCl. A single dbse of 10 mg/kg p.o. or 3 mg/kg i.v. was administered to groups of female CD1 mice (3 mice for each compound per time point). At 0 min, 4 min, 15 min, 1 h, 4 h, and 24 h groups were sacrificed and the blood and the brain were collected from each mouse for further analysis.

The brain samples were snap frozen in liquid nitrogen and stored at −20° C. A minimum of 0.2 mL of whole blood from each animal was collected in tubes containing ethylene diamine tetra-acetic acid (EDTA) as anticoagulant, thoroughly mixed, and stored at −20° C.

### Pharmacokinetic Analysis—Protocol 2

To prepare the dosing solution, 5 mg test compound was dissolved in 100 μL ethanol resulting in a compound solution of 50 mg/mL. The solution was then diluted to 2 mg/mL by adding approximately 2.4 mL 0.15 M NaCl (0.9% w/v saline), 5% v/v Tween 20 and 5% v/v PEG 400 (final ethanol conc. 4% v/v).

A single dose of 10 mg/kg p.o. or 2 mg/kg i.v. of test compound at a concentration of 10 mg/kg p.o or 2 mg/kg i.v. was administered to groups of 3 female Balb C mice. At 5 min, 15 min, 60 min, 4 h, 8 h and 24 h, groups were sacrificed and whole blood samples of approximately 0.2 mL were retrieved in EDTA to give a final concentration of 0.5 mM, additionally the brains were removed. Both whole blood and brains were snap frozen in liquid nitrogen and stored at −20° C. until shipment on solid carbon dioxide for analysis

### Analysis of the Pharmacokinetic Study Samples:

Analysis was performed by ASI Limited, (St George's Hospital Medical School, London). The concentration of the test compound in the blood and brain samples supplied was determined by HPLC with MS detection. Control, test compound free, blood samples were obtained from Harlan Sera-Lab Limited, (Loughborough, England). Time zero brain samples were supplied as control, test compound free, brain samples.

### Preparation of Brain Samples:

One hemisphere of each brain was homogenized with 5 mL water.

### Extraction of the Samples

The control or test sample of mouse brain or blood (0.05 mL), internal standard solution (0.1 mL), 5% Zinc sulphate solution (0.5 mL), and acetone (0.5 mL) were pipetted into a 2 mL polypropylene tube (Sarstedt Limited, Beaumont Leys, Leicester, UK) and the contents were then mixed for a minimum of 5 minutes (IKA-Vibrax-VXR mixer, Merck (BDH) Limited, Poole Dorset, UK). The tubes were then centrifuged in a microfuge for a minimum of 2 minutes. The solvent layer was decanted into a 4.5 mL polypropylene tube containing sodium hydroxide (0.1M, 0.1 mL) and methyl-tert-butyl ether (MTBE, 2 mL). The tube was then mixed for a minimum of 5 minutes (IKA-Vibrax-VXR mixer) and then centrifuged at 3500 rpm for 5 minutes. The solvent layer was transferred to a 4.5 mL conical polypropylene tube, placed in a SpeedVac® and evaporated to dryness.

The dried extracts were reconstituted with 0.15 mL 80% methanol and mixed for a minimum of 5 minutes (IKA-Vibrax-VXR mixer) and centrifuged at 3500 rpm for 5 minutes. The extract was transferred to auto sampler tubes (NLG Analytical, Adelphi Mill, Bollington, Cheshire, UK) and placed into the auto-sampler tray which was set at ambient temperature. The auto-sampler was programmed to inject a 0.03 mL aliquot of each extract onto the analytical column.

### Example 1

### Fermentation and Isolation of the Test Compounds

1.1 Fermentation and Isolation of 39-desmethoxyrapamycin

39-Desmethoxyrapamycin was produced by growing cultures of S. hygroscopicus MG2-10 [IJMNOQLhis] and feeding with cyclohexanecarboxylic acid (CHCA) as described below.

S. hygroscopicus MG2-10 [IJMNOQLhis] was produced by introducing into the MG2-10 strain described in WO 2004/007709 a plasmid containing the genes rapI, rapJ, rapM, rapN, rapO, rapQ and rapL. The gene cassette was constructed with the rapL gene containing a 5′ in-frame histidine tag. As described in WO 2004/007709 the plasmid also contained an origin of transfer and an apramycin resistance marker for transformation of MG2-10 by conjugation and selection of exconjugants and a phiBT1 attachment site for site-specific integration into the chromosome. Isolation of each of these genes and the method used for construction of gene cassettes containing combinations of post-PKS genes was performed as described in WO 2004/007709.

### Liquid Culture

A vegetative culture of S. hygroscopicus MG2-10 [IJMNOQLhis] was cultivated as described in Materials & Methods. Production cultures were inoculated with vegetative culture at 0.5 mL into 7 mL medium 3 in 50 mL tubes. Cultivation was carried out for 7 days, 26° C., 300 rpm. One millilitre samples were extracted 1:1 acetonitrile with shaking for 30 min, centrifuged 10 min, 13,000 rpm and analysed and quantified according to analysis Method B (see Materials & Methods). Confirmation of product was determined by mass spectrometry using analysis Method C (see Materials & Methods).

The observed rapamycin analogue was proposed to be the desired 39-desmethoxyrapamycin on the basis of the analytical data described under characterisation below.

### Fermentation

A primary vegetative culture in Medium 4 of S. hygroscopicus MG2-10 [IJMNOQLhis] was cultivated essentially as described in Materials & Methods. A secondary vegetative culture in Medium 4 was inoculated at 10% v/v, 28° C., 250 rpm, for 24 h. Vegetative cultures were inoculated at 5% v/v into medium 5 (see Materials & Methods) in a 20 L fermenter. Cultivation was carried out for 6 days at 26° C., 0.5 vvm. ≧30% dissolved oxygen was maintained by altering the impeller tip speed, minimum tip speed of 1.18 ms⁻¹ maximum tip speed of 2.75 ms⁻¹. The feeding of cyclohexanecarboxylic acid was carried out at 24 and 48 hours after inoculation to give a final concentration of 2 mM.

### Extraction and Purification

The fermentation broth (30 L) was stirred with an equal volume of methanol for 2 hours and then centrifuged to pellet the cells (10 min, 3500 rpm). The supernatant was stirred with Diaion® HP20 resin (43 g/L) for 1 hour and then filtered. The resin was washed batchwise with acetone to strip off the rapamycin analogue and the solvent was removed in vacuo. The aqueous concentrate was then diluted to 2 L with water and extracted with EtOAc (3×2 L). The solvent was removed in vacuo to give a brown oil (20.5 g).

The extract was dissolved in acetone, dried onto silica, applied to a silica column (6×6.5 cm diameter) and eluted with a stepwise gradient of acetone/hexane (20%-40%). The rapamycin analogue-containing fractions were pooled and the solvent removed in vacuo. The residue (2.6 g) was further chromatographed (in three batches) over Sephadex LH20, eluting with 10:10:1 chloroform/heptane/ethanol. The semipurified rapamycin analogue (1.7 g) was purified by reverse phase (C18) preparative HPLC using a Gilson HPLC, eluting a Phenomenex 21.2×250 mm Luna 5 μm C18 BDS column with 21 mL/min of 65% acetonitrile/water. The most pure fractions (identified by analytical HPLC, Method B) were combined and the solvent removed in vacuo to give 39-desmethoxyrapamycin (563 mg).

### Characterisation

The ¹H NMR spectrum of 39-desmethoxyrapamycin was equivalent to that of a standard (P. Lowden, Ph.D. Dissertation, University of Cambridge, 1997).

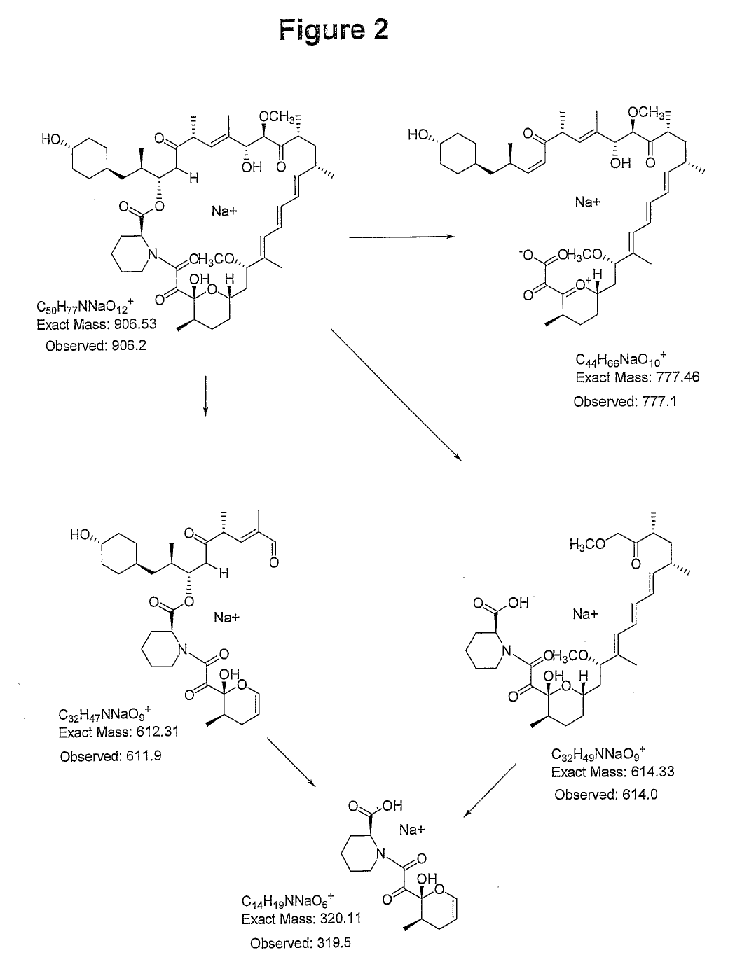

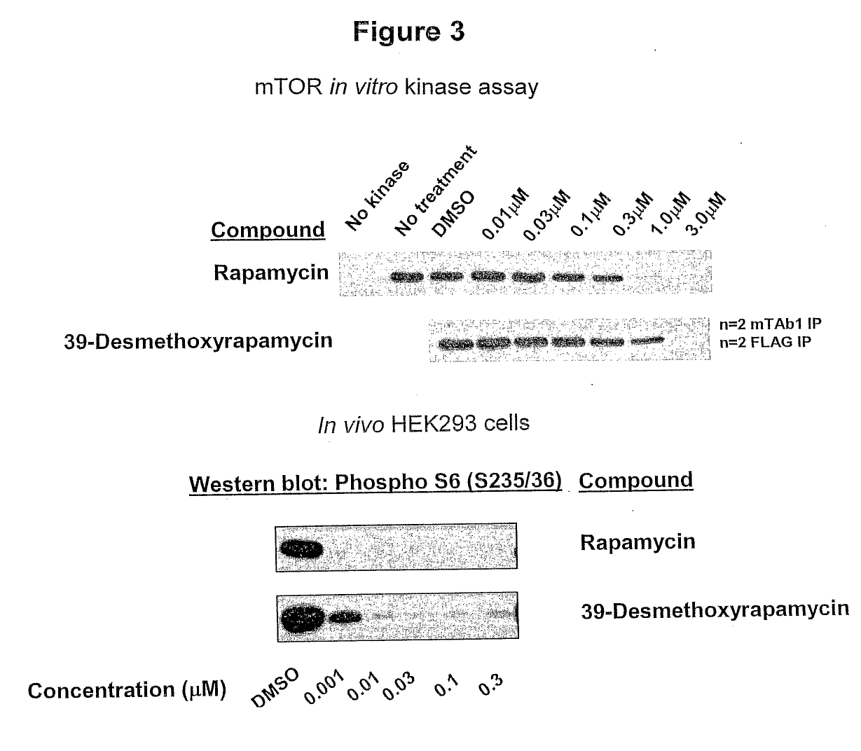

LCMS and LCMSⁿ analysis of culture extracts showed that the m/z ratio for the rapamycin analogue is 30 mass units lower than that for rapamycin, consistent with the absence of a methoxy group. Ions observed: [M−H] 882.3, [M+NH₄]⁺ 901.4, [M+Na]⁺ 906.2, [M+K]⁺ 922.2. Fragmentation of the sodium adduct gave the predicted ions for 39-desmethoxyrapamycin following a previously identified fragmentation pathway (FIG. 2) (J. A. Reather, Ph.D. Dissertation, University of Cambridge, 2000). This mass spectrometry fragmentation data narrows the region of the rapamycin analogue where the loss of a methoxy has occurred to the fragment C28-C42 that contains the cyclohexyl moiety.

These mass spectrometry fragmentation data are entirely consistent with the structure of 39-desmethoxyrapamycin

1.2 Fermentation and Isolation of 27-O-desmethyl-39-desmethoxyrapamycin

27-O-Desmethyl-39-desmethoxyrapamycin was produced by growing cultures of S. hygroscopicus MG2-10 [JMNOLhis] and feeding with cyclohexanecarboxylic acid (CHCA) as described below.

S. hygroscopicus MG2-10 [JMNOLhis] was produced by introducing into the MG2-10 strain described in WO 2004/007709 a plasmid containing the genes, rapJ, rapM, rapN, rapO, and rapL. The gene cassette was constructed with the rapL gene containing a 5′ in-frame histidine tag. As described in WO 2004/007709 the plasmid also contained an origin of transfer and an apramycin resistance marker for transformation of MG2-10 by conjugation and selection of exconjugants and a phiBT1 attachment site for site-specific integration into the chromosome. Isolation of each of these genes and the method used for construction of gene cassettes containing combinations of post-PKS genes was performed as described in WO 2004/007709.

### Liquid Culture

A vegetative culture of S. hygroscopicus MG2-10 [JMNOLhis] was cultivated as described in Materials & Methods. Production cultures were inoculated with vegetative culture at 0.5 mL into 7 mL medium 3 in 50 mL tubes. Cultivation was carried out for 7 days, 26° C., 300 rpm. One millilitre samples were extracted 1:1 acetonitrile with shaking for 30 min, centrifuged 10 min, 13,000 rpm and analysed and quantified according to analysis Method B (see Materials & Methods). Confirmation of product was determined by mass spectrometry using analysis Method C (see Materials & Methods).

The observed rapamycin analogue was proposed to be the desired 27-O-desmethyl-39-desmethoxyrapamycin on the basis of the analytical data described under characterisation below.

### Fermentation

A primary vegetative culture in Medium 2 of S. hygroscopicus MG2-10 [JMNOLhis] was cultivated essentially as described in Materials & Methods. A secondary vegetative culture in Medium 2 was inoculated at 10% v/v, 28° C., 250 rpm, for 24 h. Vegetative cultures were inoculated at 10% v/v into medium 5 (see Materials & Methods) in a 20 L fermenter. Cultivation was carried out for 6 days at 26° C., 0.75 vvm. ≧30% dissolved oxygen was maintained by altering the impeller tip speed, minimum tip speed of 1.18 ms⁻¹ maximum tip speed of 2.75 m⁻¹. The feeding of cyclohexanecarboxylic acid was carried out at 24 and 48 hours after inoculation to give a final concentration of 2 mM.

### Extraction and Purification

The fermentation broth (15 L) was stirred with an equal volume of methanol for 2 hours and then centrifuged to pellet the cells (10 min, 3500 rpm). The supernatant was stirred with Diaion® HP20 resin (43 g/L) for 1 hour and then filtered. The resin was washed batchwise with acetone to strip off the rapamycin analogue and the solvent was removed in vacuo. The aqueous concentrate was then diluted to 2 L with water and extracted with EtOAc (3×2 L). The solvent was removed in vacuo to give a brown oil (12 g).

The extract was dissolved in acetone, dried onto silica, applied to a silica column (4×6.5 cm diameter) and eluted with a stepwise gradient of acetone/hexane (20%-40%). The rapamycin analogue-containing fractions were pooled and the solvent removed in vacuo. The residue (0.203 g) was enriched by reverse phase (C18) preparative HPLC using a Gilson HPLC, eluting a Phenomenex 21.2×250 mm Luna 5 μm C18 BDS column with 21 mL/min of 65% acetonitrile/water. The most pure fractions (identified by analytical HPLC, Method B) were combined and the solvent removed in vacuo to give residue (25.8 mg). The residue was purified by reverse phase (C18) preparative HPLC using a Gilson HPLC, eluting a Hypersil 4.6×150 mm 3 μm C18 BDS column with 1 mL/min of 60% acetonitrile/water. The most pure fractions (identified by analytical HPLC, Method B) were combined and the solvent removed in vacuo to give 27-O-desmethyl-39-desmethoxyrapamycin (19.9 mg).

### Characterisation

The ¹H and 13C NMR spectra are consistent with the structure for 27-O-desmethyl-39-desmethoxyrapamycin and assignments are shown in Table 3 below.

TABLE 3
NMR data of 27-O-desmethyl-39-desmethoxyrapamycin in CDCl₃
at 500 MHz for ¹H-NMR and 125 for ¹³C-NMR.

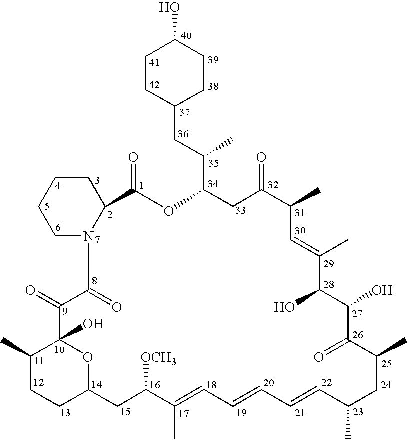

| | ¹H-NMR | | | | |
|---|---|---|---|---|---|
| | | Multiplicity, | | ¹³C-NMR | HMBC correlations |
| Position | δ ppm | Hz | COSY | δ ppm | ¹H to ¹³C |
| 1 | — | — | | 169.3 | — |
| 2 | 5.21 | br. d, 5 | H-3 | 51.3 | C-1, C-3, C-4, C-6 & C-8 |
| 3 | 2.30 | m, complex | H-2, H-4 | 27.0 | C-1, C-2, C-4 & C-5 |
| 4 | 1.78 | m, complex | H-3, H-5 | 20.7 | C-2, C-3, C-5, & C-6 |
| | 1.43 | m, complex | | | |
| 5 | 1.67 | m, complex | H-4, H-6 | 25.1 | C-3, C-4, & C-6 |
| | 1.36 | m, complex | | | |
| 6 | 3.50 | ddd, 16, 10.5, | H-5 | 46.3 | C-2, C-4, C-5, & C-8 |
| | 3.30 | 5 | | | |
| | | ddd, 16, 9.5, 6 | | | |
| 7 | — | — | | N | — |
| 8 | — | — | | 166.5 | — |
| 9 | — | — | | 194.2 | — |
| 10 | — | — | | 98.5 | — |
| 11 | 2.02 | m, complex | H-11CH₃, | 32.0 | C-9, C-10, C-12, C-13 & |
| | | | H-12 | | 11-CH₃ |
| 11-CH₃ | 0.91 | d, 6.5 | H-11 | 16.0 | C-10, C-11, & C-12 |
| 12 | 1.61 | m, complex | H-11, H-13 | 26.8 | C-10, C-11, C-13, C-14 & |
| | | | | | 11-CH₃ |
| 13 | 1.66 | m, complex | H-12, H-14 | 30.5 | C-1, C-3, C-4, C-6 & C-8 |
| | 1.43 | m, complex | | | |
| 14 | 3.95 | m, complex | H-13, H-15 | 70.8 | C-11, C-12, C-14 & C-15 |
| 15 | 1.83 | m, complex | H-14, H-16 | 35.1 | C-13, C-14, C-16, & C-17 |
| | 1.44 | m, complex | | | |
| 16 | 4.11 | dd, 5.5, 5.5 | H-15 | 83.6 | C-1, C-3, C-4, C-6 & C-8 |
| 16-OCH₃ | 3.11 | br.s | — | 55.9 | C-16, C-15 & C-17 |
| 17 | — | — | — | 135.6 | — |
| 17-CH₃ | 1.77 | s | — | 13.3 | C-16, C-17 & C-18 |
| 18 | 6.09 | d, 11 | H-19 | 130.1 | C-16, C-17, C-19, C-20 & |
| | | | | | 17-CH₃ |
| 19 | 6.35 | dd, 14.5, 11 | H-18, H-20 | 126.8 | C-17, C-18, C-20 & C-21 |
| 20 | 6.24 | dd, 14.5, 10.5 | H-19, H-21 | 132.8 | C-18, C-19, C-21 & C-22 |
| 21 | 5.99 | dd, 15, 10.5 | H-20, H-22 | 128.2 | C-19, C-20, C-22 & C-23 |
| 22 | 5.48 | dd, 15, 8 | H-21, H-23 | 137.0 | C-20, C-21, C-23, C-24 & |
| | | | | | 23-CH₃ |
| 23 | 2.29 | m, complex | H-22, 23- | 35.2 | C-21, C-22, C-24, C-25 & |
| | | | CH₃, H-24 | | 23-CH₃ |
| 23-CH₃ | 0.97 | d, 6.5 | H-23 | 21.0 | C-22, C-23 & C-24 |
| 24 | 1.87 | m, complex | H-23, H-25 | 35.1 | C-22, C-23, C-25, C-26, |
| | 1.16 | m, complex | | | 23-CH₃ & 25-CH₃ |
| 25 | 2.52 | m, complex | H-24, 25- | 40.7 | C-23, C-24, C-26, C-27 & |
| | | | CH₃ | | 25-CH₃ |
| 25-CH₃ | 0.83 | d, 6.5 | H-25 | 14.0 | C-24, C-25 & C-26 |
| 26 | — | — | — | 214.9 | — |
| 27 | 3.97 | d, 4 | H-28 | 77.8 | C-25, C-26, C-28, C-29 & |
| | | | | | 27-OCH₃ |
| 27-OH | 3.32 | s | — | O | C-27 |
| 28 | 4.34 | d, 4 | H-27 | 75.6 | C-26, C-27, C-29, C-30 & |
| | | | | | 29-CH₃ |
| 29 | — | — | — | 138.9 | — |
| 29-CH₃ | 1.66 | s | — | 13.9 | C-28, C-29 & C-30 |
| 30 | 5.39 | d, 11 | H-31 | 125.2 | C-28, C-29, C-31, C-32, |
| | | | | | 29-CH₃ & 31-CH₃ |
| 31 | 3.62 | dq, 11, 6.5 | H-30, | 44.2 | C-29, C-30, C-32, C-33 & |
| | | | 31-CH₃ | | 31-CH₃ |
| 31-CH₃ | 1.00 | d, 6.5 | H-31 | 15.8 | C-30, C-31 & C-32 |
| 32 | — | — | — | 208.4 | — |
| 33 | 2.70 | dd, 17.5, 5.5 | H-34 | 40.5 | C-31, C-32, C-34 & C-35 |
| | 2.52 | dd, 17.5, 4 | | | |
| 34 | 5.10 | ddd, 7, 5.5, 4 | H-33, H-35 | 67.3 | C-1, C-32, C-33, C-35, |
| | | | | | C-36 & 35-CH₃ |
| 35 | 1.90 | m, complex | H-34, 35- | 34.1 | C-33, C-34, C-36, C-37 & |
| | | | CH₃, H-36 | | 35-CH₃ |
| 35-CH₃ | 0.84 | d, 6.5 | H-35 | 15.2 | C-34, C-35 & C-36 |
| 36 | 1.44 | m, complex | H-35, H-37 | 39.6 | C-34, C-35, C-37, C-38, |
| | 1.20 | m, complex | | | C-42 & 35-CH₃ |
| 37 | 1.35 | m, complex | complex | 39.0 | C-35, C-36, C-38, C-39, |
| | | | | | C-41 & C-42 |
| 38 | 1.46- | m, complex | complex | 33.6* | — |
| | 0.69 | | | | |
| 39 | 1.46- | m, complex | complex | 40.7 | — |
| | 0.69 | | | | |
| 40 | 3.99 | m, complex | complex | 75.5 | C-38, C-39, C-41 & C-42 |
| 41 | 1.46- | m, complex | complex | 40.8 | — |
| | 0.69 | | | | |
| 42 | 1.46- | m, complex | complex | 33.6* | — |
| | 0.69 | | | | |

*Value showed as double integration as compared with others ¹³C-value in ¹³C-NMR spectrum.
The stereochemistry has not been determined, as we needed more NMR experiments (such as 1D and 2D NOESY) as this cause in methylene axial and equatorial ¹H has not been assigned.

LCMS and LCMSⁿ analysis of culture extracts showed that the m/z ratio for the rapamycin analogue is 44 mass units lower than that for rapamycin, consistent with the absence of a methyl and methoxy group. Ions observed: [M−H] 868.7, [M+NH₄]⁺ 887.8, [M+Na]⁺ 892.8. Fragmentation of the sodium adduct gave the predicted ions for 27-O-desmethyl-39-desmethoxyrapamycin following a previously identified fragmentation pathway (FIG. 2) (J. A. Reather, Ph.D. Dissertation, University of Cambridge, 2000). This mass spectrometry fragmentation data narrows the region of the rapamycin analogue where the loss of a methoxy has occurred to the fragment C28-C42 that contains the cyclohexyl moiety and narrows the region of the rapamycin analogue where the loss of an 0-methyl has occurred to the fragment C15-C27.

These mass spectrometry fragmentation data are entirely consistent with the structure of 27-O-desmethyl-39-desmethoxyrapamycin.

1.3 Fermentation and Isolation of 16-O-desmethyl-27-O-desmethyl-39-desmethoxyrapamycin

16-O-Desmethyl-27-O-desmethyl-39-desmethoxyrapamycin was produced by growing cultures of S. hygroscopicus MG2-10 [IJNOLhis] and feeding with cyclohexanecarboxylic acid (CHCA) as described below.

S. hygroscopicus MG2-10 [IJNOLhis] was produced by introducing into the MG2-10 strain described in WO 2004/00709 a plasmid containing the genes rapI, rapJ, rapN, rapO, and rapL. The gene cassette was constructed with the rapL gene containing a 5′ in-frame histidine tag. As described in WO 2004/007709 the plasmid also contained an origin of transfer and an apramycin resistance marker for transformation of MG2-10 by conjugation and selection of exconjugants and a phiBT1 attachment site for site-specific integration into the chromosome. Isolation of each of these genes and the method used for construction of gene cassettes containing combinations of post-PKS genes was performed as described in WO 2004/007709.

### Liquid Culture

A vegetative culture of S. hygroscopicus MG2-10 [IJNOLhis] was cultivated as described in Materials & Methods. Production cultures were inoculated with vegetative culture at 0.5 mL into 7 mL medium 3 in 50 mL tubes. Cultivation was carried out for 7 days, 26° C., 300 rpm. One millilitre samples were extracted 1:1 acetonitrile with shaking for 30 min, centrifuged 10 min, 13,000 rpm and analysed and quantified according to analysis Method B (see Materials & Methods). Confirmation of product was determined by mass spectrometry using analysis Method C (see Materials & Methods).

The observed rapamycin analogue was proposed to be the desired 16-O-desmethyl-27-O-desmethyl-39-desmethoxyrapamycin on the basis of the analytical data described under characterisation below.

### Fermentation

A primary vegetative culture in Medium 2 of S. hygroscopicus MG2-10 [IJNOLhis] was cultivated for 3 days essentially as described in Materials & Methods. A secondary vegetative culture in Medium 2 was inoculated at 10% v/v, 28° C., 250 rpm, for 48 h and a tertiary culture was inoculated at 10% v/v, 28° C., 250 rpm, for 24 h. Vegetative cultures were inoculated at 10% v/v into medium 5 (see Materials & Methods) in 3×7 L fermenters. Cultivation was carried out for 6 days at 26° C., 0.75 vvm. ≧30% dissolved oxygen was maintained by altering the impeller tip speed, minimum tip speed of 0.94 ms⁻¹ maximum tip speed of 1.88 ms⁻¹. The feeding of cyclohexanecarboxylic acid was carried out at 24 after inoculation to give a final concentration of 1 mM. L-lysine was fed at t=0.

### Extraction and Purification

The fermentation broth (12 L) was stirred with an equal volume of methanol for 2 hours and then centrifuged to pellet the cells (10 min, 3500 rpm). The supernatant was stirred with Diaion® HP20 resin (43 g/L) for 1 hour and then filtered. The resin was washed batchwise with acetone to strip off the rapamycin analogue and the solvent was removed in vacuo. The aqueous concentrate was then diluted to 2 L with water and extracted with EtOAc (3×2 L). The solvent was removed in vacuo to give a brown oil (8.75 g).

The extract was dissolved in acetone, dried onto silica, applied to a silica column (4×6.5 cm diameter) and eluted with a stepwise gradient of acetone/hexane (20%-40%). The rapamycin analogue-containing fractions were pooled and the solvent removed in vacuo. The residue (0.488 g) was further chromatographed (in three batches) over Sephadex LH20, eluting with 10:10:1 chloroform/heptane/ethanol. The rapamycin analogue-containing fractions were pooled and the solvent removed in vacuo. The semipurified rapamycin analogue (162 mg) was purified by reverse phase (C18) preparative HPLC using a Gilson HPLC, eluting a Phenomenex 21.2×250 mm Luna 5 μm C18 BDS column with 21 mL/min of 65% acetonitrile/water. The most pure fractions (identified by analytical HPLC, Method B) were combined and the solvent removed in vacuo to give 16-O-desmethyl-27-O-desmethyl-39-desmethoxyrapamycin (44.7 mg).

### Characterisation

LCMS and LCMSⁿ analysis of culture extracts showed the presence of a new rapamycin analogue eluting much earlier than all other 39-desmethoxy analogues. The m/z ratio for the various ions of the rapamycin analogue is 58 mass units lower than that for rapamycin, consistent with the absence of two O-methyl and a methoxy group. Ions observed: [M−H]⁻ 854.7, [M+NH₄]⁺ 877.8, [M+Na]⁺ 892.7, [M+K]⁺ 908.8. Fragmentation of the sodium adduct gave the predicted ions for 16-O-desmethyl-27-O-desmethyl-39-desmethoxyrapamycin following a previously identified fragmentation pathway (FIG. 2) (J. A. Reather, Ph.D. Dissertation, University of Cambridge, 2000). This mass spectrometry fragmentation data narrows the region of the rapamycin analogue where the loss of a methoxy has occurred to the fragment C28-C42 that contains the cyclohexyl moiety and narrows the region of the rapamycin analogue where the loss of the O-methyl groups has occurred to the fragment C15-C27. These NMR and mass spectrometry fragmentation data are entirely consistent with the structure of 16-O-desmethyl-27-O-desmethyl-39-desmethoxyrapamycin.

### Example 2

### In Vitro Bioassays for Anticancer Activity

In Vitro Evaluation of Anticancer Activity of 39-desmethoxyrapamycin

In vitro evaluation of 39-desmethoxyrapamycin for anticancer activity in a panel of 12 human tumour cell lines in a monolayer proliferation assay was carried out as described as Protocol 1 in the general methods above using a modified propidium iodide assay.

The results are displayed in Table 4 below, each result represents the mean of duplicate experiments. Table 5 shows the IC50 and IC70 for the compounds and rapamycin across the cell lines tested.

TABLE 4

| Test/Control (%) at | |
|---|---|
| drug concentration | |
| | 39- |
| | desmethoxy- |
| Rapamycin | rapamycin |

| Cell line | 1 μM | 10 μM | 1 μM | 10 μM |
|---|---|---|---|---|
| SF268 | 53.5 | 46 | 57.5 | 23 |
| 251L | 75.5 | 40 | 86 | 32.5 |
| H460 | 67 | 66 | 71 | 55.5 |
| MCF7 | 68.5 | 26.5 | 92.5 | 18.5 |
| MDA231 | 67 | 63.5 | 68 | 37.5 |
| MDA468 | 56.5 | 32 | 65 | 13.5 |
| 394NL | 45 | 44 | 48 | 40.5 |
| OVCAR3 | 69 | 69.5 | 77.5 | 62 |
| DU145 | 50.5 | 54 | 65.5 | 44.5 |
| LNCAP | 61 | 34 | 74.5 | 28.5 |
| A498 | 58.5 | 48.5 | 62.5 | 43.5 |
| 1138L | 42 | 21.5 | 52 | 9.5 |

TABLE 5

| | Rapamycin | 39-desmethoxyrapamycin |
|---|---|---|
| Mean IC₅₀ (microM) | 3.5 | 3.25 |
| Mean IC₇₀ (microM) | 9.1 | 6.95 |

In Vitro Evaluation of Multi-Drug Resistant (MDR) Selective Anticancer Activity of 39-desmethoxyrapamycin

In vitro evaluation of 39-desmethoxyrapamycin for selective MDR anticancer activity in the NCI 60 cell line panel of human tumour cell lines in a monolayer proliferation assay was carried out as described in Protocol 2, Materials & Methods using an SRB based assay. The results are displayed in Table 6 below:

TABLE 6
In vitro activity against high MDR-expressing cell lines

| | Log GI₅₀ | | | | |
|---|---|---|---|---|---|
| | NSCLC | Colon | CNS | Renal | Renal |
| Compound | HOP-62 | SW-620 | SF295 | A498 | UO-31 |
| 39-desmethoxyrapamycin | −8.3 | −8.3 | −5.85 | −7.07 | −8.3 |
| rapamycin | −6.63 | −4.60 | −7.0 | −6.60 | −7.0 |

It can be seen that with the exception of one cell line, 39-desmethoxyrapamycin has equivalent or improved efficacy against high MDR-expressing cell lines when compared to rapamycin.

### Example 3

### In Vitro ADME Assays

### Caco-2 Permeation Assay

Confluent Caco-2 cells (Li, A. P., 1992; Grass, G. M., et al., 1992, Volpe, D. A., et al., 2001) in a 24 well Corning Costar Transwell format were provided by In Vitro Technologies Inc. (IVT Inc., Baltimore, Md., USA). The apical chamber contained 0.15 mL Hank's balanced buffer solution (HBBS) pH 7.4, 1% DMSO, 0.1 mM Lucifer Yellow. The basal chamber contained 0.6 mL HBBS pH 7.4, 1% DMSO. Controls and tests were incubated at 37° C. in a humidified incubator, shaken at 130 rpm for 1 h. Lucifer Yellow permeates via the paracellular (between the tight junctions) route only, a high Apparent Permeability (Pₐₚₚ) for Lucifer Yellow indicates cellular damage during assay and all such wells were rejected. Propranolol (good passive permeation with no known transporter effects) & acebutalol (poor passive permeation attenuated by active efflux by P-glycoprotein) were used as reference compounds. Compounds were tested in a uni- and bi-directional format by applying compound to the apical or basal chamber (at 0.01 mM). Compounds in the apical or basal chambers were analysed by HPLC-MS (Method A, see Materials & Methods). Results were expressed as Apparent Permeability, Pₐₚₚ, (nm/s) and as the Flux Ratio (A to B versus B to A).

$$Papp ( nm \text{/} s ) = \frac{Volume Acceptor}{Area \times [ donor ]} \times \frac{Δ [ acceptor ]}{Δ time}$$

Volume Acceptor: 0.6 ml (A>B) and 0.15 ml (B>A)
Area of monolayer: 0.33 cm2
Δtime: 60 min

A positive value for the Flux Ratio indicates active efflux from the apical surface of the cells.

### Human Liver Microsomal (HLM) Stability Assay

Liver homogenates provide a measure of a compounds inherent vulnerability to Phase I (oxidative) enzymes, including CYP450s (e.g. CYP2C8, CYP2D6, CYP1A, CYP3A4, CYP2E1), esterases, amidases and flavin monooxygenases (FMOs).

The half life (T½) of test compounds was determined, on exposure to Human Liver Microsomes, by monitoring their disappearance over time by LC-MS. Compounds at 0.001 mM were incubated at for 40 min at 37° C., 0.1 M Tris-HCl, pH 7.4 with human microsomal sub-cellular fraction of liver at 0.25 mg/mL protein and saturating levels of NADPH as co-factor. At timed intervals, acetonitrile was added to test samples to precipitate protein and stop metabolism. Samples were centrifuged and analysed for parent compound using analytical Method A (see Materials & Methods).

TABLE 7
In vitro ADME Assay results

| | Compound | | | |
|---|---|---|---|---|
| | | | 16-O-des- | |
| | | | methyl-27-O- | 27-O-des- |
| | | 39-des- | desmethyl-39- | methyl-39- |
| | | methoxy | desmethoxy- | desmethoxy |
| Test | Rapamycin | rapamycin | rapamycin | rapamycin |
| Caco-2: | 2 | 29 | 13 | 4 |
| Papp (nm/s) | | | | |
| Efflux Ratio | 458 | 15 | 37 | 91 |
| HLM stability: | 40 | 59 | 47 | 27 |
| T½ min | | | | |

### Example 4

### In Vitro Binding Assays

### FKBP12

FKBP12 reversibly unfolds in the chemical denaturant guandinium hydrochloride (GdnHCl) and the unfolding can be monitored by the change in the intrinsic fluorescence of the protein (Main et al, 1998). Ligands which specifically bind and stabilise the native state of FKBP12 shift the denaturation curve such that the protein unfolds at higher concentrations of chemical denaturant (Main et al, 1999). From the difference in stability, the ligand-binding constant can be determined using equation 1.

$$Δ {G}_{app} = Δ {G}_{D - N}^{{H}_{2} O} + RT ln ( 1 + \frac{[ L ]}{{K}_{d}} ) \qquad ( 1 )$$

where ΔGₐₚₚ is the apparent difference in free energy of unfolding between free and ligand-bound forms, ΔG_{D-N}^{H}^{₂}^{O} is the free energy of unfolding in water of free protein, [L] the concentration of ligand and k_{d} the dissociation constant for the protein-ligand complex (Meiering et al, 1992). The free energy of unfolding can be related to the midpoint of the unfolding transition using the following equation:

ΔG_{D-N}^{H}^{₂}^{O}=m_{D-N}[D]_{50%} (2)

where m_{D-N} is a constant for a given protein and given denaturant and which is proportional to the change in degree of exposure of residues on unfolding (Tanford 1968 and Tanford 1970), and [D]_{50%} is the concentration of denaturant corresponding to the midpoint of unfolding. We define ΔΔG_{D-N}^{L}, the difference in the stability of FKBP12 with rapamycin and unknown ligand (at the same ligand concentration), as:

ΔΔG_{D-N}^{L}=<m_{D-N}>Δ[D]_{50%} (3)

where <m_{D-N}> is the average m-value of the unfolding transition and Δ[D]_{50%} the difference in midpoints for the rapamycin-FKBP12 unfolding transition and unknown-ligand-FKBP12 complex unfolding transition. Under conditions where [L]>K_{d}, then, ΔΔG_{D-N}, can be related to the relative K_{d}s of the two compounds through equation 4:

$$Δ Δ {G}_{D - N}^{L} = RT ln \frac{{K}_{d}^{X}}{{K}_{d}^{rap}} \qquad ( 4 )$$

where K_{d} ^{rap} the dissociation constant for rapamycin and K_{d}^{X} is the dissociation constant for unknown ligand X. Therefore,

$${K}_{d}^{X} = {K}_{d}^{rap} exp ( \frac{〈 {m}_{D - N} 〉 {Δ [ D ]}_{50 \%}}{RT} ) \qquad ( 5 )$$

For the determination of the K_{d} of 39-desmethoxyrapamycin, the denaturation curve was fitted to generates values for m_{D-N} and [D]_{50%}, which were used to calculate an average m-value, <m_{D-N}>, and Δ[D]_{50%}, and hence K_{d}^{X}. The literature value of K_{d}^{rap} of 0.2 nM is used.

TABLE 8
In vitro FKBP12 binding assay results

| | FKBP12 |
|---|---|
| | K_{d} (nM) |
| rapamycin | 0.2 |
| 39-desmethoxyrapamycin | 0.7 |
| 27-O-desmethyl-39-desmethoxyrapamycin | 0.8 |
| 16-O-desmethyl-27-O-desmethyl-39-desmethoxy rapamycin | 101 |
| 27-desmethoxy-39-desmethoxy rapamycin | 160 |

mTOR

Inhibition of mTOR can be established indirectly via the measurement of the level of phosphorylation of the surrogate markers of the mTOR pathway and p70S6 kinase and S6 (Brunn et al., 1997; Mothe-Satney et al., 2000; Tee and Proud, 2002; Huang and Houghton, 2002).

HEK293 cells were co-transfected with FLAG-tagged mTOR and myc-tagged Raptor, cultured for 24 h then serum starved overnight. Cells were stimulated with 100 nM insulin then harvested and lysed by 3 freeze/thaw cycles. Lysates were pooled and equal amounts were immunoprecipitated with FLAG antibody for the mTOR/Raptor complex. Immunoprecipitates were then processed: samples treated with compound (0.00001 to 0.003 mM) were pre-incubated for 30 min at 30° C. with FKBP12/rapamycin, FKBP12/39-desmethoxyrapamycin or vehicle (DMSO), non-treated samples were incubated in kinase buffer. Immunoprecipitates were then subject to in vitro kinase assay in the presence of 3 mM ATP, 10 mM Mn2+ and GST-4E-BP1 as substrate. Reactions were stopped with 4× sample buffer then subjected to 15% SDS-PAGE, wet transferred to PVDF membrane then probed for phospho-4E-BP1 (T37/46).

Alternatively, HEK293 cells were seeded into 6 well plates and pre-incubated for 24 h and then serum starved overnight. Cells were then pre-treated with vehicle or compound for 30 min at 30° C., then stimulated with 100 nM insulin for 30 min at 30° C. and lysed by 3 freeze/thaw cycles and assayed for protein concentration. Equal amounts of protein were loaded and separated on SDS-PAGE gels. The protein was then wet transferred to PVDF membrane and probed for phospho-S6 (S235/36) or phospho-p70 S6K (T389).

The results of these experiments are summarised as FIG. 3

### Example 5

### In Vitro P-gp Substrate Assay

Cell Lines

The cell lines used in the present study (MACL MCF7 and MACL MCF7 ADR) were both provided by the National Cancer Institute, USA.

Cells were routinely passaged once or twice weekly. They were maintained in culture for no more than 20 passages. All cells were grown at 37° C. in a humidified atmosphere (95% air, 5% CO₂) in RPMI 1640 medium (PAA, Cölbe, Germany) supplemented with 5% fetal calf serum (PAA, Cölbe, Germany) and 0.1% Gentamicin (PAA, Cölbe, Germany).

### Assay Protocol

A modified propidium iodide assay based on protocol 1 described above was used to assess the effects of 39-desmethoxyrapamycin (Dengler et al, 1995). Briefly, cells were harvested from exponential phase cultures by trypsination, counted and plated in 96 well flat-bottomed microtiter plates at a cell density of 5.000 cells/well. After a 24 h recovery to allow the cells to resume exponential growth, 0.01 mL of Verapamil at a concentration of 0.18 mg/mL or 0.01 mL culture medium were added to the cells in order to yield a final concentration of Verapamil in the wells of 0.01 mg/mL. This concentration was found in previous experiments to be non-toxic to the cells. Culture medium containing 39-desmethoxyrapamycin, taxol or culture medium alone (for the control wells) was added at 0.01 mL per well. The compounds were applied in triplicates in 8 concentrations in half log steps ranging from 0.03 mM down to 10 nM. Following 3 days of continuous drug exposure, medium or medium with compound was replaced by 0.2 mL of an aqueous propidium iodide (PI) solution (7 mg/L). Since PI only passes leaky or lysed membranes, DNA of dead cells will be stained and measured, while living cells will not be stained. To measure the proportion of living cells, cells were permeabilized by freezing the plates, resulting in death of all cells. After thawing of the plates, fluorescence was measured using the Cytofluor 4000 microplate reader (excitation 530 nm, emission 620 nm), giving a direct relationship to the total cell number. Growth inhibition was expressed as Test/Control×100 (% TIC). Assays were only considered evaluable if the positive control (Taxol) induced a shift in tumor growth inhibition in the presence and absence of Verapamil and if vehicle treated control cells had a fluorescence intensity >500.

Preparation of 39-desmethoxyrapamycin Testing Solutions

A stock solution of 3.3 mM of 39-desmethoxyrapamycin was prepared in DMSO and stored at −20° C. The stock solution was then thawed on the day of use and stored at room temperature prior and during dosing. The dilution steps were carried out using RPMI 1640 medium and to result in solutions of 18-fold the final concentration.

### Results

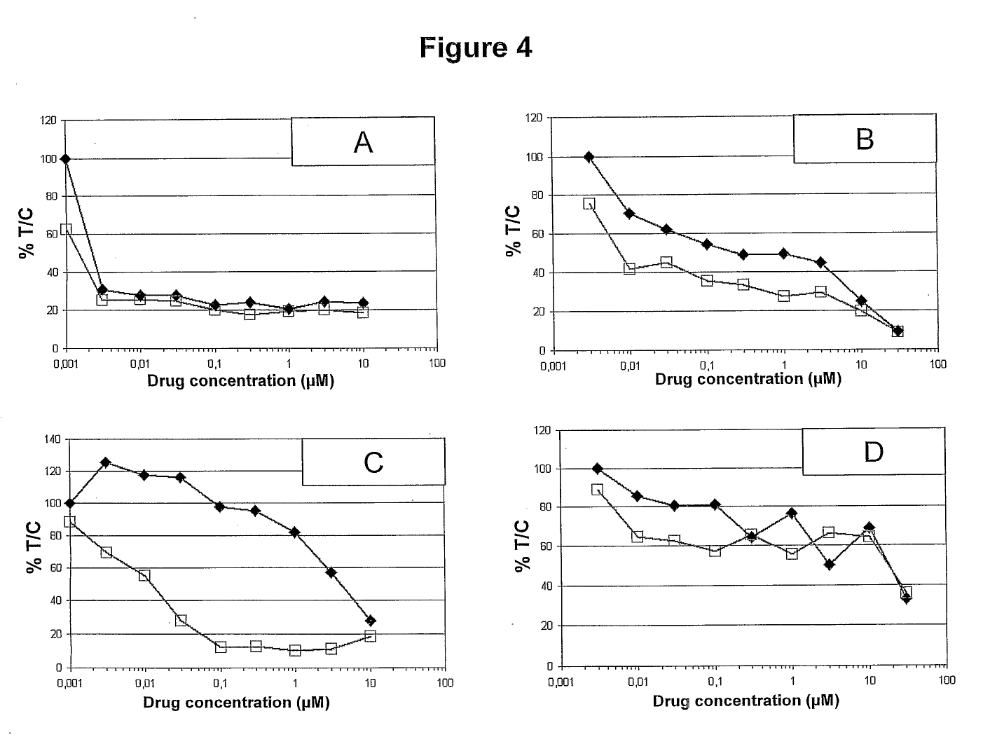

FIG. 4 shows four graphs demonstrating the % T/C values at all test concentrations for paclitaxel (A and C) and 39-desmethoxyrapamycin (B and D) in normal (A and B) or high P-gp expressing (C and D) cell lines. The filled diamonds represent the values after the administration of paclitaxel or 39-desmethoxyrapamycin alone, the open squares represent the values after the administration of paclitaxel or 39-desmethoxyrapamycin in the presence of 0.01 mg/mL Verapamil (a P-gp inhibitor).

Paclitaxel, a known P-gp substrate showed reduced potency in inhibiting P-gp expressing cancer cell line MCF7 ADR and this reduced potency was restored by the co-administration of verapamil, a P-gp inhibitor (FIGS. 4A and 4C).

39-desmethoxyrapamycin did not show a significant shift in the growth proliferation curves in the P-gp expressing cell line MCF7 ADR either with or without verapamil (FIGS. 4B and 4D) demonstrating that 39-desmethoxyrapamycin is not a substrate for P-gp.

### Example 6

### Pharmacokinetic Analysis

6.1 PK Analysis of Rapamycin and 39-desmethoxyrapamycin

Pharmacokinetic analysis using the standard methods as described above was performed for rapamycin and 39-desmethoxyrapamycin, (the protocol used for each compound is indicated in Table 9).

The AUC for each compound in blood or in brain tissue was calculated using Kinetica 4.4 (InnaPhase Corporation), using a non-compartmental model and the trapezoidal method for AUC calculation.

The partition coefficient (Rᵢ) for each compound after p.o. and i.v. administration was calculated as shown below:

$${R}_{i} = \frac{A U {C}_{BRAIN}}{A U {C}_{BLOOD}}$$

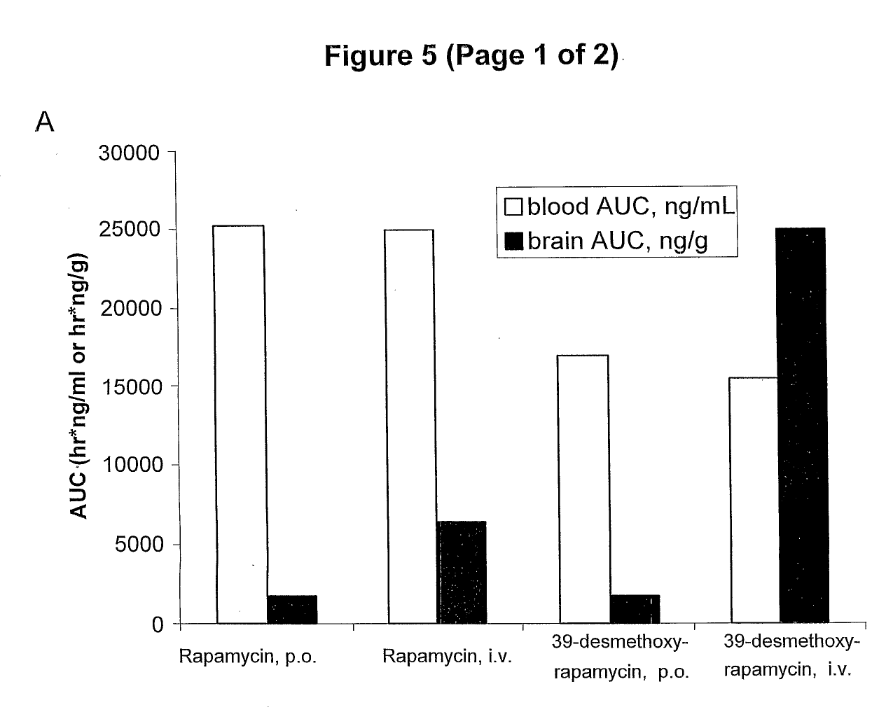

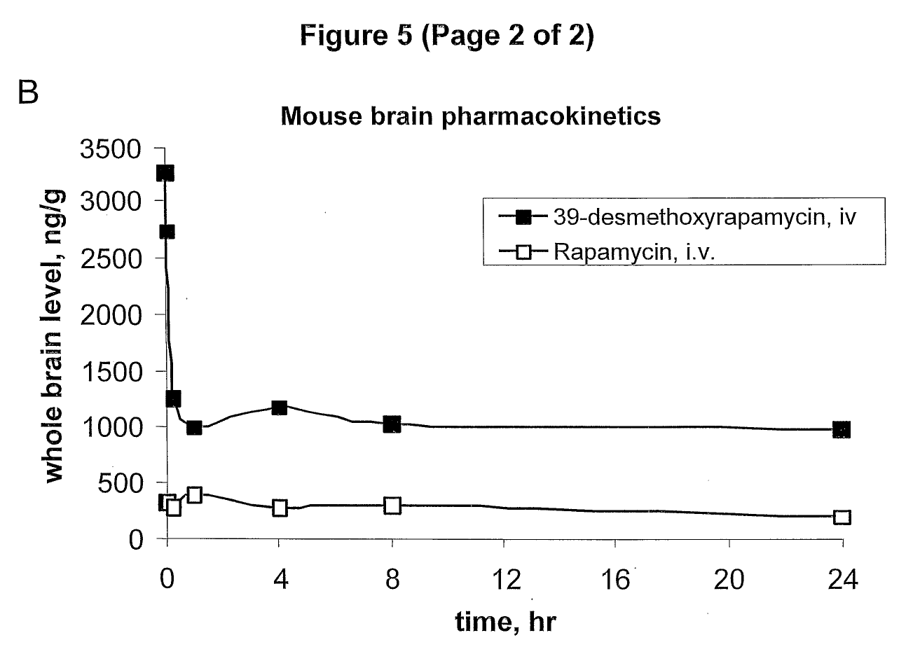

The results of this analysis are summarised in Table 9 below and in FIG. 5.

TABLE 9
Summary of pharmacokinetic data

| | PK | AUC_{BRAIN} | | AUC_{BLOOD} | | Rᵢ | |
|---|---|---|---|---|---|---|---|
| Compound | protocol | p.o. | i.v. | p.o. | i.v. | p.o. | i.v. |
| Rapamycin | 2 | 1658.37 | 6338.11 | 25212.2 | 24876.5 | 0.066 | 0.255 |
| 39-desmethoxyrapamycin | 1 | 1697.69 | 24911.4 | 16856.5 | 15444.3 | 0.100 | 1.613 |

6.2 PK Analysis of Rapamycin, 39-desmethoxyrapamycin and 27-O-desmethyl-39-desmethoxy Rapamycin

Pharmacokinetic analysis using the standard methods as described above was performed for rapamycin, 39-desmethoxyrapamycin and 27-O-desmethyl-39-desmethoxy rapamycin, (using Protocol 1 described above).

The AUC for each compound in blood or in brain tissue was calculated using Kinetica 4.4 (InnaPhase Corporation), using a non-compartmental model and the trapezoidal method for AUC calculation.

The partition coefficient (R) for each compound after i.v. administration was calculated as shown below:

$${R}_{i} = \frac{A U {C}_{BRAIN}}{A U {C}_{BLOOD}}$$

TABLE 10
Pharmacokinetic data

| Compound | AUC_{BRAIN}, i.v. | AUC_{BLOOD}; i.v. | Rᵢ; i.v. |
|---|---|---|---|
| Rapamycin | 12156.6 | 10756.2 | 1.13 |
| 39-desmethoxyrapamycin | 15543.9 | 8017.88 | 1.94 |
| 27-O-desmethyl-39- | 8440.05 | 1851.12 | 4.56 |
| desmethoxy rapamycin | | | |

### Example 7

### Activity in the Experimental Allergic Encephalomyelitis (EAE) Model of Multiple Sclerosis

Experimental allergic encephalomyelitis (EAE) is an autoimmune inflammatory and demyelinating disease of the central nervous system (CNS), and is considered the best available animal counterpart for multiple sclerosis (MS). The disease can be induced in genetically susceptible animals by the injection of whole spinal cord, or myelin basic protein (MBP) in complete Freund's adjuvant (CFA). The antigen-specific effector cells involved in the CNS damage are class II major histocompatibility complex (MHO) restricted CD4⁺ T lymphocytes. Recently, the role of cytokines such as interleukin-1 (IL-1), tumor necrosis factors (TNF) or interferons (IFN) in inflammatory responses has received increasing attention. Upon activation by antigen, T cells produce several lymphokines which in the case of EAE, may be directly or indirectly responsible for the CNS damage. The lymphokines likely to be involved in the pathogenesis of EAE are IL-2, IFN-γ and TNF-β. IL-2 has an important role in T cell activation and proliferation, while IFN-γis a potent mediator of macrophage activation. In addition, IFN-γ induces the production of inflammatory cytokines such as IL-1. TNF, and also the expression of class II MHC molecules, among others, on the endothelial cells of blood vessels in the CNS, and on astrocytes, which are thought to play an important role in antigen presentation to encephalitogenic T cells.

### 7.1—Animals and Immunization Procedure

Eight to 10 week-old male Lewis rats were kept under standard laboratory conditions (non specific pathogen free) with free access to food and water. EAE was induced by a single injection into the base of the tail of 50 mL Freund's incomplete adjuvant (Difco, Detroit, Mich.) plus 50 mL saline containing 25 mg guinea pig spinal cord and 1 mg Mycobacterium tuberculosis strain H 37 RA (Difco).

### 7.2—Clinical and Histological Scoring

Rats were examined every day by measuring their body weights and clinical signs of EAE until 30 days after immunization. These clinical gradings were carried out by an observer unaware of the treatment: 0=no illness, 1=flaccid tail, 2=moderate paraparesis, 3=severe paraparesis, 4=moribund state, 5=death. End of the disease was defined as complete absence of clinical symptoms and return to motility of the preimmunization period, with the rat being graded 0 for 5 consecutive days.

### 7.3—Experimental Treatment

Test compounds were be given at different doses (5 or 15 mg/kg bd wt) under both a prophylactic and therapeutic regime. For the prophylactic part of the study the treatment was started one day prior to immunization, and for the therapeutic part of the study it was initiated on day 7 post immunization (p.i.). Vehicle-treated rats treated under the same experimental conditions, either prophylactically or therapeutically, as were used for controls. Treatment was given p.o. daily six times a week until day 30 p.i. Cyclophosphamide was used as a positive control.

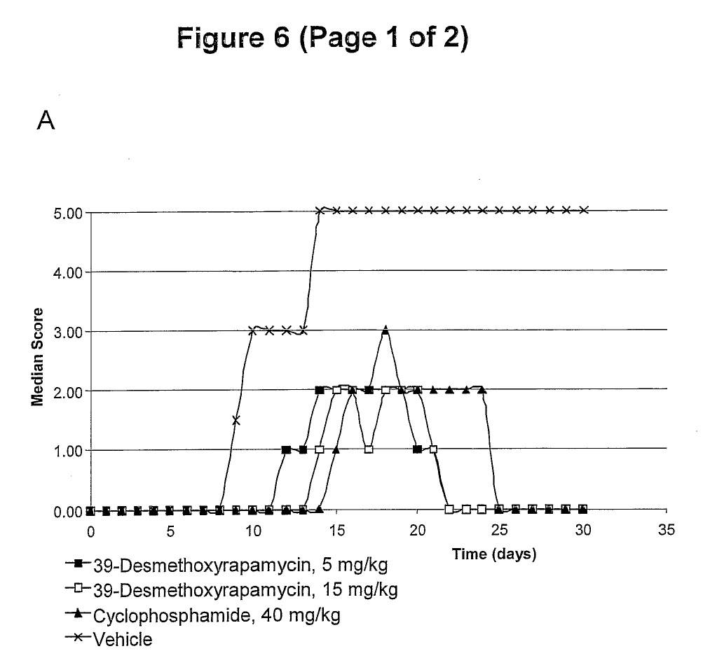

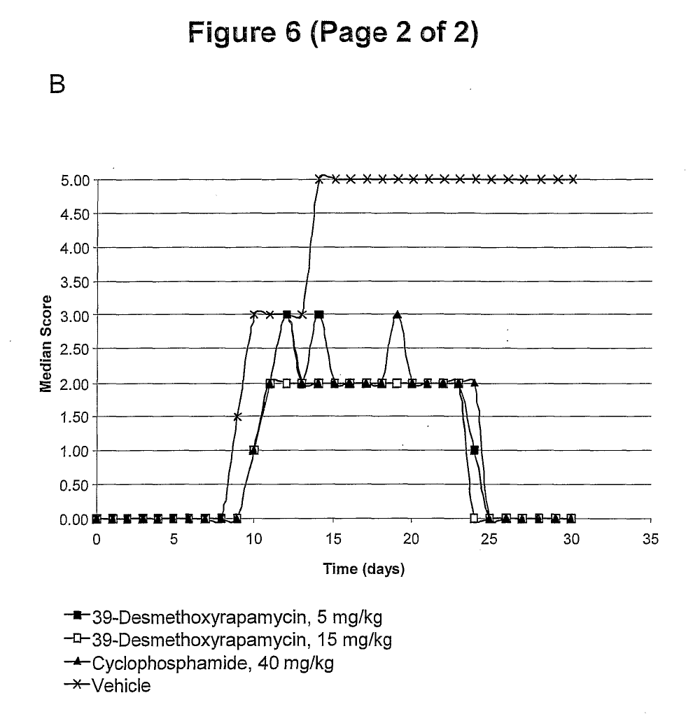

The results of the experiment are shown in FIG. 6 and in Table 11 below. FIG. 6A shows the effect of the prophylactic regime of 39-desmethoxyrapamycin at 5 and 15 mg/kg, FIG. 6B shows the effect of the therapeutic regime of 39-desmethoxyrapamycin at 5 and 15 mg/kg. For each regime the effects of 40 mg/kg cyclophosphamide are shown as a positive control. In both graphs the median score of each group is shown. It can be seen that 39-desmethoxyrapamycin has equivalent efficacy in this model to cyclophosphamide and that it reduces not only the severity of symptoms but also reduces the duration of the episode. It should be noted that due to the death during the study of 5 out of 7 vehicle-treated rats, the median value for this group remained at 5, however, the two surviving rats did both eventually return to baseline values by day 28.

TABLE 11

| | | | | Duration | Cumulative |
|---|---|---|---|---|---|
| | Dose, | | Onset | (days) | score |
| Compound | mg/kg | Regime | Mean ± St Dev. | Mean ± St Dev. | Mean ± St Dev. |
| Vehicle | n/a | n/a | 9 ± 0.8 | 21 ± 1.3 | 84 ± 28.6 |
| 39- | 5 | Prophylactic | 12 ± 2.1* | 10 ± 1.8* | 17 ± 4.6* |
| desmethoxyrapamycin | | | | | |
| 39- | 15 | Prophylactic | 12 ± 2.4* | 10 ± 3.4* | 22 ± 22* |
| desmethoxyrapamycin | | | | | |
| Cyclophosphamide | 40 | Prophylactic | 13 ± 2.6* | 13 ± 3.4* | 33 ± 14.1* |
| 39- | 5 | Therapeutic | 10 ± 1.0* | 15 ± 1.3* | 30 ± 3.1* |
| desmethoxyrapamycin | | | | | |
| 39- | 15 | Therapeutic | 9 ± 1.4* | 16 ± 3.3* | 32 ± 5.7* |
| desmethoxyrapamycin | | | | | |
| Cyclophosphamide | 40 | Therapeutic | 11 ± 1.1* | 15 ± 3.2* | 37 ± 26.6* |

*statistically different from the vehicle-treated control, p < 0.05, Mann Whitney Rank Sum test.

### Example 8

### Antitumor Activity Study of 39-desmethoxyrapamycin in a Model of Glioma Orthotopically Xenografted in Nude Mice

### 8.1—Preparation for Study

### 8.1.1—Preparation of Samples:

The test compound was dissolved in ethanol (0.027 mL/mg compound) and vortexed for 20 min until the solution was clear. Ethanolic solutions were aliquoted as appropriate and stored at −20° C. The ethanolic solution was then made up to the correct concentration with vehicle (4% Ethanol, 5% Tween-20, 5% polyethyleneglycol 400 in 0.15 M NaCl, prepared with sterile endotoxin free components where possible).

### 8.1.2—Means of Administration

The test substance and control vehicle were administered intravenously (IV, bolus) by injection into the caudal vein of the test mice. An injection volume of 10 mL/kg was used, based on the most recent body weight of mice.

### 8.1.3—Cancer Cell Line

The cell line used for the study was U87-MG, a glioblastoma cell line initiated by J. Ponten from a grade III glioblastoma from a 44 year-old female Caucasian (Poten et al., 1968).

### 8.1.4—Cell Culture Conditions for Establishment of the Cell Line.

Tumor cells were grown as a monolayer at 37° C. in a humidified atmosphere (5% CO₂, 95% air). The culture medium was RPMI 1640 (Ref. BE12-702F, Cambrex) containing 2 mM L-glutamine supplemented with 10% fetal bovine serum (Ref. DE14-801E, Cambrex). The cells were adherent to plastic flasks. For experimental use, tumor cells were detached from the culture flask by 5 minutes treatment with trypsin-versene (Ref. BE17-161E, Cambrex), in Hanks' medium without calcium or magnesium (Ref. BE10-543F, Cambrex). The cells were counted in a hemocytometer and their viability was assessed by 0.25% trypan blue exclusion.

### 8.2—Induction of Glioma by Stereotaxic Injection in the Brain of Nude Mice

Mice were stereotaxically injected with U87-MG cells at D, 24 to 48 hours after a whole body irradiation with a γ-source (2.5 Gy, Co⁶⁰, INRA BRETENIERE, Dijon). For the stereotaxic injection of tumour cells, mice were anesthetised by an intraperitoneal injection of Ketamine 100 mg/kg (Ketamine500®, Ref 043KET204, Centravet, France) and Xylazine 5 mg/kg (Rompun®, Ref 002ROM001, Centravet, France) in 0.9% NaCl solution at 10 mL/kg/inj. Cells were stereotaxically injected using 3 independent stereotaxic apparatus (Kopf Instrument, Germany and Stoelting Company, USA) in the right frontal lobe with 1×10⁵ U87-MG tumor cells re-suspended in 0.002 mL of RPMI-1640 medium. 0.002 mL of the cell suspension were injected at 500 nL/min.

### 8.3—Treatment Schedule

At D7, mice were weighed and randomized according to their individual body weight into 3 groups of mice. Four (4) additional mice were added to each treatment group for MRI imaging. The groups were selected such that the mean body weight of each group was not statistically different from the others (analysis of variance). Test substances were administered as defined below.
8.3.1 Mice from group 1 received 5 cycles of daily IV injections of test substances vehicle for 3 consecutive days (at D7 to D9, D14 to D16, D21 to D23, D28 to D30 and D35 to D37: (Q1D×3)×5W). Each cycle was separated by a 4-day period of wash out
8.3.2 Mice from group 2 received 5 cycles of daily IV injections of 39-desmethoxyrapamycin at 3 mg/kg/inj for 3 consecutive days at D7 to D9, D14 to D16, D21 to D23, D28 to D30 and D35 to D37: (Q1D×3)×5W). Each cycle was separated by a 4-day period of wash out
8.3.3 Mice from group 3 were not treated.
   The treatment schedule is summarized in table 12 below:

TABLE 12

| | Number of | | | Dose |
|---|---|---|---|---|
| Group | animals | Treatment | Route | (mg/kg/inj) |
| 1 | 9 (+4) | Vehicle | IV | — |
| 2 | 10 (+4) | 39-desmethoxyrapamycin | IV | 3 |
| 3 | 16 | Untreated | n/a | n/a |

### 8.4—MRI Analysis

MRI analysis of the brain was performed at D23 and D37. All the MRI analyses were performed at 4.7T in the Pharmascan magnet (Bruker, Wissembourg). Mice were positioned within the dedicated mouse cradle and the 38 mm diameter cylindrical coil under continuous anesthesia with isoflurane.

After tripilot acquisitions, a turboRare T2 weighted sequence was performed. Acquisitions covered the entire brain including the tumour. The tumour volume was determined by manually drawing a region of interest (ROI) around the tumour in each slice and by summation of all the surfaces.

### 8.5—Results

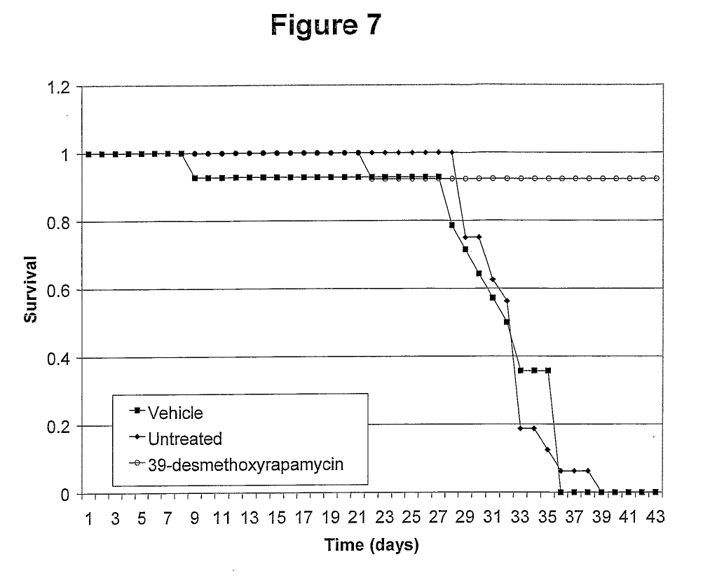

FIG. 7 shows the survival graph for each treatment group until day 43.

Additionally, the results were expressed as a percent (TIC %) where T represents the median survival times of animals treated with 39-desmethoxyrapamycin and C represents the median survival times of control animals treated with vehicle. TIC % was calculated as follows:

T/C %=[T/C]×100

Additionally the MRI analysis was used to calculate the average calculated tumour volume per treatment group, the results are summarised in Table 13 below. As all the vehicle-treated animals had died by day 37 it was not possible to compare tumour sizes at this stage.

TABLE 13

| Group | Day 23 (mm³) |
|---|---|
| Vehicle | 18.75 |
| 39-desmethoxyrapamycin | 1.25 |

Each data point represents the mean of 4 values.

### REFERENCES

Alvarez, M., Paull, K., Monks, A., Hose, C., Lee, J. S., Weinstein, J., Greyer, M., Bates, S., Fojo, T., (1995). “Generation of a drug resistance profile by quantitation of mdr-1/P-glycoprotein in the cell lines of the National Cancer Institute Anticancer Drug Screen”, Journal of Clinical Investigation, 95, 2205-2214.
An, W. L., R. F. Cowburn, et al. (2003). “Up-regulation of phosphorylated/activated p70 S6 kinase and its relationship to neurofibrillary pathology in Alzheimer's disease.” Am J Pathol 163(2): 591-607.
Avramut, M. and Achim, C. L. (2002). “Immunophilins and their ligands: insights into survival and growth of human neurons.” Physiol Behav 77(4-5): 463-8.
Baker, H., Sidorowicz, A., Sehgal, S. N., and Vézina, C. (1978) “Rapamycin (AY-22,989), a new antifungal antibiotic. III. In vitro and in vivo evaluation”. Journal of Antibiotics 31, 539-545.
Boyd, M. R. and Paull, K. D., (1995). “Some Practical Considerations and Applications of the National Cancer Institute In Vitro Anticancer Drug Discovery Screen”, Drug Development Research 34, 91-109,
Bradl M., Hohlfeld R. (2003), “Molecular pathogenesis of neuroinflammation”. J Neurol Neurosurg Psychiatry. 74:1364-70.

Brunn, G. J., Fadden, P., Haystead, T. A., Lawrence, J. C. Jr. (1997) “The mammalian target of rapamycin phosphorylates sites having a (Ser/Thr)-Pro motif and is activated by antibodies to a region near its COOH terminus”, J Biol. Chem. 272(51), 32547-32550.
Brunn, G. J., Williams, J., Sabers, C., Wiederrecht, G., Lawrence, J. C., and Abraham, R. T. (1996) “Direct inhibition of the signaling functions of the mammalian target of rapamycin by the phosphoinositide 3-kinase inhibitors, wortmannin and LY294002”. EMBO Journal 15: 5256-5267.
Chang, S. M., P. Wen, at al. (2005). “Phase II study of CCI-779 in patients with recurrent glioblastoma multiforme.” Invest New Drugs 23(4): 357-61.
Crowe, A., Bruelisauer, A., Duerr, L., Guntz, P., Lemaire, M., (1999), “Absorption and intestinal metabolism of SDZ-RAD and rapamycin in rats”. Drug Metab Dispos. 27(5), 627-32.
Cummins, C. L., Jacobsen, W., Christians, U., Benet, L. Z., (2004) “CYP3A4-Transfected Caco-2 Cells as a Tool for Understandinf Biochemical Absorption Barriers: Studies with Sirolimus and Midazolam”, The Journal of Pharmacology, 308(1), 143-155
Dancey, J. E., (2002), “Clinical development of mammalian target of rapamycin inhibitors” Hematol Oncol Clin N Am, 16, 1101-1114.
Dengler W. A., Schulte J., Berger D. P., Mertelsmann R. and Fiebig H H. (1995) “Development of a propidium iodide fluorescence assay for proliferation and cytotoxicity assay”. Anti-Cancer Drugs, 6, 522-532.
Dumont, F. J. and Su, Q. X. (1995). “Mechanism of action of the immunosuppressant rapamycin”. Life Sciences 58(5): 373-395.
Emborg M. E., (2004) “Evaluation of animal models of Parkinson's disease for neuroprotective strategies” J Neurosci Methods, 139(2):121-43;
Fiebig H. H., Dengler W. A. and Roth T. (1999) “Human tumor xenografts: Predictivity, characterization, and discovery of new anticancer agents”. In: Fiebig H H, Burger A M (eds). Relevance of Tumor Models for Anticancer Drug Development. Contrib. Oncol., 54: 29-50.
Findlay J. A, and Radics, L. (1980) Canadian Journal of Chemistry 58:579.
Gallant-Haidner H. L., Trepanier D. J., Freitag D. G., Yatscoff R. W. 2000, “Pharmacokinetics and metabolism of sirolimus”, Ther Drug Monit. 22(1), 31-5.

German D. C. and Eisch A. J., (2004), “Mouse models of Alzheimer's disease: insight into treatment”, Rev Neurosci, 15:353-69.

Grass, G. M., Rubas, W., Jezyk, N., (1992) “Evaluation of CACO-2 monolayers as a predictor of drug permeability in colonic tissues”. FASEB Journal, 6, A1002.
Gregory M. A., Gaisser S, Lill R. E., Hong H, Sheridan R. M., Wilkinson B, Petkovic H, Weston A. J., Carletti I, Lee H. L., Staunton J, Leadlay P F. (2004) “Isolation and characterization of pre-rapamycin, the first macrocyclic intermediate in the biosynthesis of the immunosuppressant rapamycin by S. hygroscopicus”. Angew Chem Int Ed Engl. 43(19), 2551-3
Hersch S. M. and Ferrante R. J., (2004), “Translating therapies for Huntington's disease from genetic animal models to clinical trials”, NeuroRx. 1(3):298-306
Huang, S. and Houghton, P. J., 2002. “Mechanisms of resistance to rapamycins”. Drug Resist. Update, 4(6), 378-391.
Huang, S., Bjornsti, M. A. and Houghton P. J. (2003). “Rapamycins: mechanism of action and cellular resistance.” Cancer Biol Ther 2(3): 222-32.
Kahan, B. D., Chang, J. Y., and Sehgal, S. N. (1991) “Preclinical evaluation of a new potent immunosuppressive agent, rapamycin”. Transplantation 52: 185-191.
Kirchner, G. I., Winkler, M., Mueller L., Vidal, C., Jacobsen, W., Franzke, A., Wagner, S., Blick, S., Manns M. P., and Sewing K.-F.(2000) “Pharmacokinetics of SDZ RAD and cyclosporin including their metabolites in seven kidney graft patients after the first dose of SDZ RAD”. British Journal of Clinical Pharmacology 50:449-454.
Kuhn B., Jacobsen W., Christians U., Benet L. Z., Kollman P. A. (2001), “Metabolism of sirolimus and its derivative everolimus by cytochrome P450 3A4: insights from docking, molecular dynamics, and quantum chemical calculations”. J Med Chem. 44(12), 2027-34.
Kuo, C. J., Chung, J. K., Fiorentino, D. F., Flanagan, W. M., Blenis, J., and Crabtree, G. R. (1992) “Rapamycin selectively inhibits interleukin-2 activation of p70 S6 kinase”. Nature 358: 70-73.
Lampen A., Zhang Y., Hackbarth I., Benet L. Z., Sewing K. F., Christians U. (1998) “Metabolism and transport of the macrolide immunosuppressant sirolimus in the small intestine”. J Pharmacol Exp Ther. 285(3), 1104-12.
Langmann T., Mauerer R., Zahn A., Moehle C., Probst M., Stremmel W., Schmitz G. (2003) “Real-time reverse transcription-PCR expression profiling of the complete human ATP-binding cassette transporter superfamily in various tissues”. Clin Chem. 49(2), 230-8.
Laplante A., Demeule M., Murphy G. F., Beliveau R. (2002) “Interaction of immunosuppressive agents rapamycin and its analogue SDZ-RAD with endothelial P-gp”. Transplant Proc. 34(8), 3393-5.
Lee, J-S., Paull, K., Alvarez, M., Hose, C., Monks, A., Greyer, M., Fojo, A. T., Bates, S. E., 1994. “Rhodamine efflux patterns predict P-glycoprotein substrates in the National Cancer Institute drug screen”. Molecular Pharmacology 46,627-638.

Lee J. K., Bussey K. J., Gwadry F. G., Reinhold W., Riddick G., Pelletier S. L., Nishizuka S., Szakacs G., Annereau J. P., Shankavaram U., Lababidi S., Smith L. H., Gottesman M. M., Weinstein J. N. (2003) “Comparing cDNA and oligonucleotide array data: concordance of gene expression across platforms for the NCI-60 cancer cells”. Genome Biol. 4(12), R82.
Li, A. P. (1992) “Screening for human ADME/Tox drug properties in drug discovery”. Drug Discovery Today, 6, 357-366.
Lowden, P. A. S., (1997) Ph.D. Dissertation, University of Cambridge. “Studies on the biosynthesis of rapamycin”.
Main, E. R. G., Fulton, K. F. & Jackson, S. E. (1998). “The Context-Dependent Nature of Destabilising Mutations on the Stability of FKBP12”. Biochemistry 37, 6145-6153.
Main, E. R. G., Fulton, K. F. & Jackson, S. E. (1999). “Folding of FKBP12: Pathway of Folding and Characterisation of the Transition State”. J. Mol. Biol. 291, 429-444.
McAlpine, J. B, Swanson S. J., Jackson, M., Whittern, D. N. (1991). “Revised NMR assignments for rapamycin”. Journal of Antibiotics 44: 688-690.
Meiering, E. M., Serrano, L. & Fersht, A. R. (1992). “Effect of Active Site Residues in Barnase on Activity and Stability”. J. Mol. Biol. 225, 585-589.
Mothe-Satney, I., Brunn, G. J., McMahon, L. P., Capaldo, C. T., Abraham, R. T., Lawrence, J. C. Jr-. (2000) “Mammalian target of rapamycin-dependent phosphorylation of PHAS-I in four (S/T)P sites detected by phospho-specific antibodies”. J Biol Chem. 275(43), 33836-33843.
Paiva, N. L., Demain, A. L., and Roberts, M. F. (1991) “Incorporation of acetate, propionate, and methionine into rapamycin By Streptomyces hygroscopicus”. Journal of Natural Products 54: 167-177.
Pardridge, (2005), “The Blood-Brain Barrier: Bottleneck in Brain Drug Development”, NeuroRx, 2, 3-14
Perin, E. C., (2005), “Choosing a Drug-Eluting Stent: A Comparison Between CYPHER and TAXUS”, Reviews in Cardiovascular Medicine, 6 (suppl 1), pp S13-S21.
Persidis A. (1999), “Cancer multidrug resistance” Nat Biotechnol. 17: 94-5
Poten J. et al., (1968), “Long term culture of normal and neoplastic human glia.” Acta Pathol. Microbiol. Scand., 74: 465-86
Raught B., Gingras, A-C. and Sonenberg, N.; (2001), “The target of rapamycin (TOR) proteins”, PNAS, 98(13): 7037-7044
Ravikumar, B., R. Duden, et al. (2002). “Aggregate-prone proteins with polyglutamine and polyalanine expansions are degraded by autophagy.” Hum Mol Genet 11(9): 1107-17.
Reather, J. A., (2000), Ph.D. Dissertation, University of Cambridge. “Late steps in the biosynthesis of macrocyclic lactones”.
Roth T., Burger A. M., Dengler W., Willmann H. and Fiebig H. H. (1999) “Human tumor cell lines demonstrating the characteristics of patient tumors as useful models for anticancer drug screening”. In: Fiebig H H, Burger A M (eds). Relevance of Tumor Models for Anticancer Drug Development. Contrib. Oncol., 54: 145-156.
Sathasivam, K., C. Hobbs, at al. (1999). “Transgenic models of Huntington's disease.” Philos Trans R Soc Lond B Biol Sci 354(1386): 963-9
Sedrani, R., Cottens, S., Kallen, J., and Schuler, W. (1998) “Chemical modifications of rapamycin: the discovery of SDZ RAD”. Transplantation Proceedings 30: 2192-2194.
Sehgal, S. N., Baker, H., and Vézina, C. (1975) “Rapamycin (AY-22,989), a new antifungal antibiotic II. Fermentation, isolation and characterization”. The Journal of Antibiotics 28: 727-733.
Schober A. (2004), “Classic toxin-induced animal models of Parkinson's disease: 6-OHDA and MPTP” Cell Tissue Res, 318(1):215-24)
Stein U., Jurchott K., Schlafke M., Hohenberger P. (2002) “Expression of multidrug resistance genes MVP, MDR1, and MRP1 determined sequentially before, during, and after hyperthermic isolated limb perfusion of soft tissue sarcoma and melanoma patients”. J Clin Oncol. 20(15):3282-92.
Szakacs G., Annereau J. P., Lababidi S., Shankavaram U., Arciello A., Bussey K. J., Reinhold W., Guo Y., Kruh G. D., Reimers M., Weinstein J. N., Gottesman M. M. 2004, “Predicting drug sensitivity and resistance: profiling ABC transporter genes in cancer cells”. Cancer Cell. 6(2):129-37.
Tanford, C. (1968). “Protein Denaturation”. Adv. Prot. Chem. 23, 121-282.
Tanford, C. (1970). “Protein Denaturation. Part C. Theoretical models for the mechanism of denaturation”. Advances in Protein Chemistry 24, 1-95
Tee, A. R. and Proud, C. G. (2002) “Caspase cleavage of initiation factor 4E-binding protein 1 yields a dominant inhibitor of Cap-dependent translation and reveals a novel regulatory motif”. Mol. Cell. Biol. 22, 1674-1683
Trepanier D. J., Gallant H., Legatt D. F., Yatscoff R. W. (1998), “Rapamycin: distribution, pharmacokinetics and therapeutic range investigations: an update”. Clin Biochem. 31(5):345-51.
Vézina, C., Kudelski, A., and Sehgal, S. N. (1975) “Rapamycin (AY-22,989), a new antifungal antibiotic. I. Taxonomy of the producing streptomycete and isolation of the active principle”. The Journal of Antibiotics 28: 721-726.
Volpe, D. A., Faustino, P. J., Yu, L. X., (2001) “Towards standardisation of an in vitro method of drug absorption”. Pharmacopeial Forum, 27, 2916-2922.
Yu, K., Toral-Barza, L., Discafani, C., Zhang, W. G., Skotnicki, J., Frost, P., Gibbons, J. J. (2001) “mTOR, a novel target in breast cancer: the effect of CCI-779, an mTOR inhibitor, in preclinical models of breast cancer”. Endocrine-Related Cancer 8:249-258.

1. A method of treating a medical condition in a patient, in need thereof, comprising administration of an effective amount of a 39-desmethoxyrapamycin analogue according to Formula (I), 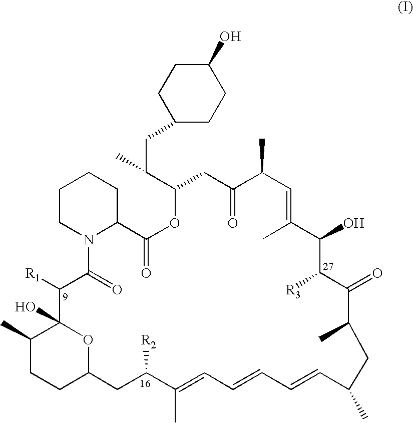
wherein, R₁ represents (H,H) or ═O and R₂ and R₃ each independently represent H, OH or OCH₃; or a pharmaceutically acceptable salt thereof, wherein said medical condition is the result of neural injury or disease or is a cancer or B-cell malignancy.

2. The method of claim 1, wherein said medical condition affects the central nervous system and requires the crossing of the blood-brain barrier.

3. The method according to claim 1, wherein the medical condition is selected from the group consisting of brain tumour(s) and neurodegenerative conditions.

4. The method of claim 3 wherein the medical condition is a brain tumour.

5. The method of claim 4, wherein the brain tumour is glioblastoma multiforme.

6. The method of claim 3, wherein the medical condition is a neurodegenerative condition.

7. The method of claim 6, wherein the neurodegenerative condition is Alzheimer's disease.

8. The method of claim 6, wherein the neurodegenerative condition is multiple sclerosis.

9. The method of claim 1, said cancer or B-cell malignancy is resistant to one or more existing anticancer agent(s).

10. The method of claim 9, wherein the cancer or B-cell malignancy expresses P-glycoprotein.

11. The method of claim 10, wherein the cancer or B-cell malignancy has a high expression level of P-glycoprotein.

12. The method of claim 1, wherein the 39-desmethoxyrapamycin analogue or a pharmaceutically acceptable salt thereof is administered intravenously.

13. The method of claim 1, further comprising administering one or more other therapeutically effective agent(s).

14. The method of claim 1 further comprising administering one or more agents selected from the group consisting of methotrexate, leukovorin, adriamycin, prenisone, bleomycin, cyclophosphamide, 5-fluorouracil, paclitaxel, docetaxel, vincristine, vinblastine, vinorelbine, doxorubicin, tamoxifen, toremifene, megestrol acetate, anastrozole, goserelin, anti-HER2 monoclonal antibody (e.g. Herceptin™), capecitabine, raloxifene hydrochloride, EGFR inhibitors, VEGF inhibitors, proteasome inhibitors, and hsp90 inhibitors.

15. The method of claim 1 wherein the 39-desmethoxyrapamycin analogue is 39-desmethoxyrapamycin.

16. The method of claim 1 wherein the 39-desmethoxyrapamycin analogue additionally differs from rapamycin at one or more of positions 9, 16 or 27.

17. The method according to claim 16, wherein the 39-desmethoxyrapamycin analogue differs from rapamycin at one or more of positions 16 or 27.

18. The method according to claim 16, wherein the 39-desmethoxyrapamycin analogue differs from rapamycin at positions 16 and 27.

19. The method according to claim 16, wherein the 39-desmethoxyrapamycin analogue has a hydroxyl group at position 27.

20. The method according to claim 16, wherein the 39-desmethoxyrapamycin analogue has a hydrogen at position 27.

21. The method according to claim 16 to 20, wherein the 39-desmethoxyrapamycin analogue has a hydroxyl group at position 16.

22. A pharmaceutical composition comprising a 39-desmethoxyrapamycin analogue according to Formula (I), 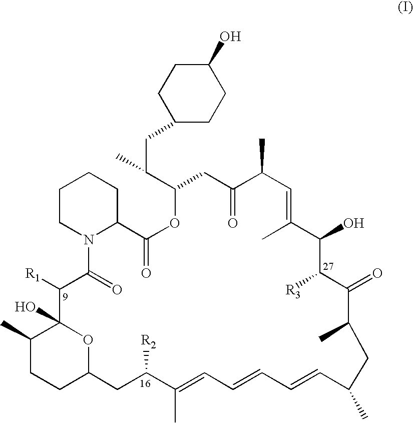
wherein, R₁ represents (H,H) or ═O and R₂ and R₃ each independently represent H, OH or OCH₃, or a pharmaceutically acceptable salt thereof, together with a pharmaceutically acceptable carrier.

23. A pharmaceutical composition according to claim 22 that is specifically formulated for intravenous administration.
